SEQUENCE LISTING (4 sequences)

SEQ ID NO: 1 - protein, 16 aa, Drosophila melanogaster
RQIKIWFQNRRMKWKK

SEQ ID NO: 2 - protein, 21 aa, Homo sapiens
AACTGGAACAGACAAACTATC

SEQ ID NO: 3 - protein, 21 aa, Homo sapiens
AAGACCAGACGCCACTCCAAC

SEQ ID NO: 4 - protein, 21 aa, Homo sapiens
GCTTGGCTTCCACAACTAGAA

(19) United States
(12) Patent Application Publication
(10) Pub. No.: US 2013/0202649 A1
(43) Pub. Date: Aug. 8, 2013

(54) ACTIVATION OF INNATE IMMUNITY FOR NUCLEAR REPROGRAMMING OF SOMATIC CELLS

(71) Applicant: The Board of Trustees of the Leland Stanford Junior University, Palo Alto, CA (US)

(72) Inventors: John P. Cooke, Palo Alto, CA (US); Ji Eun Lee, Menlo Park, CA (US); Nazish Sayed, Stanford, CA (US)

(73) Assignee: The Board of Trustees of the Leland Stanford Junior University, Palo Alto, CA (US)

(21) Appl. No.: 13/744,272

(22) Filed: Jan. 17, 2013

Related U.S. Application Data
(60) Provisional application No. 61/587,259, filed on Jan. 17, 2012.

(51) Int. Cl. C12N 5/071

(52) U.S. Cl. CPC C12N 5/0602
USPC 424/278.1; 435/377; 435/366

(57) ABSTRACT

The nuclear reprogramming of somatic cells with non-integrating factors is shown to be greatly accelerated by activation of innate immune responses in the somatic cell. Methods of activating innate immunity include activation of toll-like receptors, e.g. TLR3. Somatic cells with activated innate immune responses can be reprogrammed to induced pluripotent cells or to transdifferentiated cells.

### GOVERNMENT RIGHTS

This invention was made with Government support under contracts HL100397 and HL103400 awarded by the National Institutes of Health. The Government has certain rights in this invention.

### BACKGROUND OF THE INVENTION

Seminal studies by Yamanaka and colleagues revealed that ectopic expression of certain transcriptional factors could induce pluripotency in somatic cells. These induced pluripotent stem cells (iPSC) self-renew and differentiate into a wide variety of cell types, making them an appealing option for disease- and patient-specific regenerative medicine therapies. They have been used to successfully model human disease and have great potential for use in drug screening and patient-specific cell therapy. Furthermore, iPSCs generated from diseased cells can serve as useful tools for studying disease mechanisms and potential therapies. However, much remains to be understood about the underlying mechanisms of reprogramming of somatic cells to iPSCs, and there is concern regarding potential clinical applications in the absence of mechanistic insights.

The set of factors (RFs) for reprogramming to pluripotency include Oct3/4, Sox2, c-Myc, Klf4, Lin28, and Nanog. Oct3/4 and Sox2 are transcription factors that maintain pluripotency in embryonic stem (ES) cells while Klf4 and c-Myc are transcription factors thought to boost iPSC generation efficiency. The transcription factor c-Myc is believed to modify chromatin structure to allow Oct3/4 and Sox2 to more efficiently access genes necessary for reprogramming while Klf4 enhances the activation of certain genes by Oct3/4 and Sox2. Nanog, like Oct3/4 and Sox2, is a transcription factor that maintains pluripotency in ES cells while Lin28 is an mRNA-binding protein thought to influence the translation or stability of specific mRNAs during differentiation. Recently, it has been shown that retroviral expression of Oct3/4 and Sox2, together with co-administration of valproic acid, a chromatin destabilizer and histone deacetylase inhibitor, is sufficient to reprogram fibroblasts into iPSCs.

To generate iPSCs from somatic cells, viral vectors or plasmids have been used to overexpress some combination of these reprogramming factors. However, these methods result in a low efficiency of reprogramming and fail to provide precise control of the reprogramming process. Furthermore, these methods for nuclear reprogramming inherently raise concerns about potential tumorigenicity and gene-silencing mutations caused by DNA integration. The integration of foreign DNA into the host genome from retroviral infection raises concerns that the integration of foreign DNA could silence indispensable genes or induce dysregulation of these genes. While Cre-LoxP site gene delivery or PiggyBac transposon approaches have been used to excise foreign DNA from the host genome following gene delivery, neither strategy eliminates the risk of mutagenesis because they leave a small insert of residual foreign DNA.

As an alternative to genetic modification, cell permeant proteins (CPP) have been generated in which the reprogramming factors are fused to a domain that provides for membrane transport of the protein into the nucleus, for integration-free nuclear reprogramming. Successful reprogramming to pluripotency has been achieved by using purified recombinant proteins in murine embryonic fibroblasts. Although human cells have been reprogrammed using cell extracts from embryonic stem cells (ESCs) as well as from HEK293 cells overexpressing the four transcription factors, human cells have not yet been reprogrammed using purified CPPs. And even with mouse cells, reprogramming efficiencies with CPPs are more than 10-fold lower (˜0.001%) by comparison to those achieved with retroviral vectors (0.1%-1%).

A CPP and/or small-molecule based approach for iPSC generation or transdifferentiation to a different somatic cell type avoids all concerns for integration of foreign DNA, and provides for greater control over the concentration, timing, and sequence of factor stimulation, however significant problems have remained in the actual practice of such methods. The present invention addresses this issue.

### SUMMARY OF THE INVENTION

Compositions and methods are provided for efficient generation of induced pluripotential cells or transdifferentiated cells using non-integrating methods. In the methods of the invention, a somatic cell for which reprogramming to pluripotency or transdifferentiation is desired is contacted with an effective dose of an agonist of a toll-like receptor (TLR), which TLR include, without limitation, TLR3. The contacting step may be performed before, concurrently with, or following contact of the cell with non-integrating reprogramming factors, usually concurrently. Non-integrating reprogramming factors are nuclear-acting polypeptides or small molecules that alter transcription, and which can induce reprogramming in targeted cells. In some embodiments the reprogramming factors are polypeptides fused to a polypeptide permeant domain, e.g. nona-arginine, tat, etc. as known in the art. In some embodiments the reprogramming factor is one or more of Oct3/4, Sox2, c-Myc, Klf4, Lin28, and Nanog.

In some embodiments, a reprogrammed cell population is provided, wherein an initial population of somatic cells is reprogrammed to an induced pluripotent or transdifferentiated cell population. Such cells find use in a variety of methods known in the art, including pharmacological screening, autologous or allogeneic therapeutic cell administration, and the like. The reprogrammed cell population provides for advantages due to the absence of integrative genetic factors.

In other embodiments, kits are provided for nuclear reprogramming of somatic cells. Such kits may comprise an activator of innate immunity, e.g. one or more TLR agonists, including without limitation double stranded nucleic acids, such as poly I:C. Such kits may further comprise reagents for non-integrative induction of pluripotency, for example one or a cocktail of cell-permeant proteins, e.g. SOX2, OCT4, Nanog, KLF4, cMYC, and the like. Such kits may alternatively or in combination provide one or a cocktail of factors useful in transdifferentiating cells to a lineage of interest. For example, an endothelial transdifferentiation kit may comprise one or more of BMP4, VEGF, bFGF, 8-Br-cAMP, SB431542, etc. Such kits may further comprise suitable buffers, cell growth medium, instructions and the like necessary to perform the methods of the invention.

Kits and methods are also provided for in vivo use of the methods of the invention, where a therapeutic agent comprising an activator of innate immunity, and one or more cell permeant peptides and/or small molecules is administered in vivo for therapeutic modulation of cell and/or tissue phenotype.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

FIG. 1: Different patterns of gene expression induced by reprogramming factors expressed from viral vectors or delivered as cell-permeant peptides (A) Fold-expression of Nanog following infection with retroviral expression vector (red line) or cell permeant peptide (blue line). By comparison to the viral vector pMX-Sox2, the cell permeant CPP-SOX2 causes a delayed expression of downstream target and pluripotency genes. Relative fold change in gene expression was determined following treatment with 200 nM CPPSOX2 daily or after a single pMXSox2 infection on Day 0. All data represented as mean±s.d., n=3, *P<0.005. (B) Because the temporal pattern of expression of the selected genes (Jarid2, Zic2, bMyb, Oct4, Sox2 and Nanog) was remarkably similar for each treatment condition, their change in fold-expression over time is shown as an average fold-increase of all six genes. Note that when Sox2 is presented in the form of a viral vector, target gene expression increases rapidly, by comparison to when Sox2 is presented in the form of a CPP. (C) By comparison to the viral vector pMXOct4, the cell permeant CPPOCT4 causes a delayed expression of downstream target and pluripotency genes. Nanog gene expression is representative of this different temporal pattern of gene expression. Relative fold change in gene expression was determined following treatment with 200 nM CPPOCT4 daily or after a single pMOct4 infection on Day 0. All data represented as mean±s.d., n=3, *P<0.005. (D) Summary figure showing the average fold-change in the selected genes (i.e. Tcf4, Gap43, Nanog, Sox2 and Oct4) over time for each condition. Note that when Oct4 is presented in the form of a viral vector, target gene expression increases rapidly, by comparison to when Oct4 is presented in the form of a CPP.

FIG. 2: Irrelevant retroviral vector accelerates CPP-induced gene expression (A) Relative fold change in gene expression levels of Nanog following pMX-Sox2 (red line), CPP-SOX2 (blue line) or pMX-GFP+CPP-SOX2 (green line) treatments. (B) Summary figure showing the average fold-change in the selected genes (i.e. Jarid2, Zic2, bMyb, Oct4, Sox2, and Nanog) over time for each condition. When Sox2 is given in the form of a CPP, activation of the downstream target genes is delayed. However, in the presence of an irrelevant retroviral vector, target gene expression increases rapidly, and mimics that of pMX-Sox2. (C) Relative fold change in gene expression levels of Nanog following pMX-Oct4, CPP-OCT4 or pMX-GFP+CPP-OCT4 treatments. (D) Summary figure showing the average fold-change in the selected genes (i.e. Tcf4, Gap43, Nanog, Sox2 and Oct4) over time for each condition. All data represented as mean±s.d., n=3, *P<0.005.

FIG. 3: Knockdown of TLR3 pathway inhibits action of retroviral vector encoding Oct4 (A) Gene expression of Oct4 following retroviral-Oct4 (pMX-Oct4) infection is reduced in fibroblasts treated with the TRIF-inhibitory peptide (Pepinh-TRIF). The lower panel shows a summary diagram of the average fold-changes over time in the selected pluripotent genes (Oct4, Sox2 and Nanog) in the four conditions. (B) Gene expression of Oct4 following retroviral-Oct4 (pMX-Oct4) infection is reduced in TRIF shRNA-knockdown fibroblasts. The lower panel shows a summary diagram of the average fold-changes over time in the selected pluripotent genes (Oct4, Sox2 and Nanog) in the four conditions. (C) Gene expression of Oct4 following retroviral-Oct4 (pMX-Oct4) infection is reduced in TLR3 shRNA-knockdown fibroblasts. The lower panel (D-F) shows a summary diagram of the average fold-changes over time in the selected pluripotent genes (Oct4, Sox2 and Nanog) in the four conditions. All data represented as mean±s.d., n=3, *P<0.005.

FIG. 4: TLR3-TRIF knockdown fibroblasts exhibit impaired nuclear reprogramming (A) Protocol for iPSC generation using the reprogramming factors, delivered as retroviral vectors. (B) Representative images of iPSCs on day 30 after initiation of retroviral nuclear reprogramming for scramble, MyD88, TRIF and TLR3 shRNA knockdown fibroblasts. In fibroblasts where elements of the TLR3 signaling pathway were knocked down (ie. TRIF shRNA or TLR3 shRNA cell lines), the development of human iPSC colonies was markedly delayed. By contrast, in fibroblasts where the adaptor for all other TLRs was knocked down (MyD88 shRNA), or in those fibroblasts treated with scrambled shRNA, no delay in hiPSC development was noted. (C) Total number of hiPSC colonies on day 35 in scramble, MyD88, TRIF and TLR3 shRNA knockdown fibroblast cell lines transduced by the reprogramming factors delivered by retroviral transfection. The yield of hiPSC colonies was reduced by knocking down elements of the TLR3 signaling pathway. *P<0.05; scramble compared to TRIF or TLR3 shRNA knockdown fibroblasts. (D) Fold change in Oct4 gene expression in scramble, MyD88, TRIF and TLR3 shRNA knockdown fibroblasts at day 35.

FIG. 5: Poly I:C accelerates CPP-induced target gene expression (A) Relative fold change in gene expression levels of Jarid2 following pMX-Sox2 (red line), CPP-SOX2 (blue line) or poly I:C+CPPSOX2 (green line) treatments. (B) Summary figure of these selected genes (i.e. Jarid2, Zic2, bMyb, Oct4, Sox2, and Nanog) exhibiting the temporal pattern of average gene expression following each treatment. Poly I:C markedly enhances the expression of downstream genes by CPPSOX2. (C) Relative fold change in gene expression levels of Nanog following pMX-Oct4, CPPOCT4 or Poly I:C+CPPOCT4 treatments. (D) Summary figure of these selected genes (i.e. Tcf4, Gap43, Nanog, Sox2 and Oct4) exhibiting the temporal pattern of gene expression following each treatment. Poly I:C markedly enhances the expression of downstream genes by CPPOct4. All data represented as mean±s.d., n=3, *P<0.005.

FIG. 6. TLR3 activation enhances reprogramming in a doxycycline-inducible system (A) SSEA-1 live staining showing iPSC colonies derived from MEFs expressing a dox-inducible polycistronic transgene construct encoding the four reprogramming factors, 4 wks after exposure to doxycycline. In some wells, MEFs were also exposed to a retroviral construct encoding GFP, or to poly I:C. (B) Histogram showing SSEA-1+ colonies at 2 and 3 weeks in primary plates. Co-administration of poly I:C, or a retroviral construct encoding GFP, markedly increased the yield of doxycycline-induced reprogramming. (C) Gene expression of Oct4 and Sox2 was accelerated by co-administration of poly I:C, or a retroviral construct encoding GFP.

FIG. 7. TLR3 activation stimulates CPP-induced reprogramming of human fibroblasts (A) Protocol for human iPSC generation using four CPP-TFs (OCT4-R11, SOX2-R11, KLF4-R11 and cMYC-R11). (B) Gene expression of Oct4 was increased by co-administration of poly I:C, by day 30-45. (C) TRA-1-81 positive colonies counted at day 30 and day 40 in the presence and absence of poly I:C. Co-administration of poly I:C markedly increased the yield of CPP induced reprogramming. (D) ES-like colony formation at day 32 of CPP-induced transactivation (10×)

FIG. 8. Difference in downstream gene expression pattern induced by individual reprogramming factors expressed from viral vectors or delivered as cell-permeant peptides (A) Heat-map showing the intensity of expression over time (Days 0-6) of selected pluripotent genes (Sox2, Oct4, Nanog) and Sox2-associated genes (Jarid2, Zic2, bMyb), following retroviral or CPP-SOX2 treatments, by comparison to vehicle. Vehicle or irrelevant retroviral construct (pMX-GFP) have no effect on gene expression. Retroviral Sox2 causes an early increase in expression of each of the six genes, followed by a decline. CPP-SOX2 causes a delayed increase in gene expression. (B) Heat-map showing the intensity of expression over time (Days 0-6) of selected pluripotent genes (Sox2, Oct4, Nanog) and Oct4-associated genes (Tcf4, GAP43).

FIG. 9. Irrelevant retroviral vector accelerates CPP-induced gene expression (A-B) Relative fold change in gene expression levels of Jarid2 and Zic2 following pMX-Sox2 (red line), CPP-SOX2 (blue line) or pMX-GFP+CPP-SOX2 (green line) treatments. (C) Summary figure showing the average fold-change in the selected genes over time for each condition. (D-E) Relative fold change in gene expression levels of Tcf4 and GAP43 following pMX-Oct4, CPP-OCT4 or pMX-GFP+CPP-OCT4 treatments.

FIG. 10. Non-integrating pMX-GFP accelerates gene expression induced by CPP-OCT4 (A) Immunofluorescent images of BJ fibroblasts infected with either pMX-GFP mutant (top) or pMX-GFP wt (bottom). (B-E) Relative fold change in gene expression levels of Oct4, Sox2, Nanog and TLR3 following pMX-GFP (red line), pMX-GFP+CPP-OCT4 (blue line), pMX-GFP mutant (green line) or pMX-GFP mutant+CPP-OCT4 (purple line) treatments.

FIG. 11. Retroviral GFP/OCT4/SOX2 infection stimulates innate immunity Relative fold change in gene expression levels of innate immunity related genes, STAT1, STAT2, IFNβ, NF-κB, TLR3 and TLR4 following pMX-Oct4, pMX-Sox2, pMX-GFP, pMX-GFP+CPP-OCT4, pMX-GFP+CPP-SOX2, CPP-OCT4 or CPP-SOX2.

FIG. 12. Knockdown of TLR3 signaling decreases pluripotent gene expression (Related to FIG. 3) (A) Gene expression of Tcf4, NF-κB or TLR3 following retroviral-Oct4 (pMX-Oct4) infection is reduced in fibroblasts treated with the TRIF-inhibitory peptide (Pepinh-TRIF). (B) Gene expression of Tcf4, NF-κB or TLR3 following pMX-Oct4 infection is reduced in TRIF shRNA knockdown fibroblasts. (C) Gene expression of Tcf4, NF-κB or TLR3 following pMX-Oct4 infection is reduced in TLR3 shRNA-knockdown fibroblasts.

FIG. 13. Inhibition of MyD88 does not affect pMX-Oct4 induced gene expression (A) Gene expression of Oct4, Tcf4, NF-κB or TLR3 following retroviral-Oct4 (pMX-Oct4) infection remains unaltered in fibroblasts treated with the MyD88-inhibitory peptide (Pepinh-MyD88). (B) Gene expression of Oct4, Tcf4, NF-κB or TLR3 following pMX-Oct4 infection remains unaltered in MyD88 shRNA-knockdown fibroblasts.

FIG. 14. Poly I:C enhances expression of innate immunity genes Relative fold change in gene expression levels of innate immunity related genes, STAT1, STAT2, IFNβ, NF-κB, TLR3 and TLR4 following poly I:C, poly I:C+CPP-OCT4 or poly I:C+CPP-SOX2.

FIG. 15. Poly I:C enhances CPP-induced gene expression via TLR3 (A-B) Relative fold change in gene expression levels of Zic2 and Nanog following pMX-Sox2 (red line), CPP-SOX2 (blue line) or poly I:C+CPP-SOX2 (green line) treatments. (C) Summary figure showing the average fold-change in the selected genes over time for each condition. (D, E) Relative fold change in gene expression levels of Tcf4 and GAP43 following pMX-Oct4, CPPOCT4 or poly I:C+CPP-OCT4 treatments.

FIG. 16. Reprogramming in Doxycycline-induced secondary MEF system Morphological changes in doxycycline treated MEFs. Poly I:C as well as pMX-GFP accelerated changes in the with small, compact rounded cells aggregating in the wells at 3 days. Infection with pMX-GFP accelerates colony formation, as day 7 in the viral particle infected group observed a number of small colonies. By day 14, typical mES-like colonies appeared, many of which had activated SSEA-1.

FIG. 17. Poly I:C/Viral particles promote early epigenetic modification (day 2) (A) ChIP analysis to assess H3K4me3 of the Oct4 promoters, on Day 2 of exposure to the various treatment conditions. Stimulation of TLR3 with pMX-GFP or with poly I:C had no effect, nor did CPP-SOX2. However, in combination with pMX-GFP or with poly I:C, the cell permeant peptide CPP-SOX2 mimicked the effects of retroviral Sox2 (pMX-Sox2). (B) ChIP analysis to assess H3K9me3 of the Oct4 promoters, on Day 2 of exposure to the various treatment conditions, Data represented as mean±s.d, n=2 (*P<0.005, **P<0.02) (C) Immunoblot showing HDAC 1 protein expression confirms that poly I:C induces an early (day 2) and sustained (to day 6) inhibition of HDAC1 expression. The expression of HP-1α is unaffected (although it is re-distributed, see FIGS. S11A-C)

FIG. 18. Poly I:C/Viral particles promote epigenetic modification (time course, day 2-6) (A) ChIP analysis to assess H3K4me3 of the Oct4 promoters, on Day 2, 4, and 6 of exposure to the various treatment conditions. (B) ChIP analysis to assess H3K9me3 of the Oct4 promoters, on Day 2, 4, and 6 of exposure to the various treatment conditions. (C) ChIP analysis to assess H3K4me3 of the Sox2 promoters, on Day 2, 4, and 6 of exposure to the various treatment conditions. (D) ChIP analysis to assess H3K9me3 of the Sox2 promoters, on Day 2, 4, and 6 of exposure to the various treatment conditions.

FIG. 19. Imaging analysis from confocal microscopy (A) Size of HP1α-positive spots in the presence of CPP-Sox2 with pMX-GFP or Poly I:C was increased. (B) The number of HP1α-positive spots was decreased indicating rearrangement HP1α location in the presence of CPP-Sox2 with pMX-GFP or Poly I:C. (C) Confocal microscopy of HP1α. (D) Western blot for HP-1α expression with time course (Day 2, 4 and 6)

FIG. 20. Poly I:C activates NF-κB via TLR3-TRIF signaling (A) Transcriptional expression of TLR3 and NF-κB in response of poly I:C. (B)-(C) Luciferase assay for NF-κB activity reveals that poly I:C (but not CPP-SOX2) substantially increases NF-κB activity, an effect that is inhibited by knocking down elements of the TLR3 signaling pathway.

FIG. 21: Direct reprogramming of human fibroblasts to functional endothelial cells via innate immunity activation and microenvironmental cues: (A) Protocol for direct reprogramming of human BJ fibroblasts to endothelial cells. The figure describes the time course and sequential treatments of different medias: Human fibroblasts were treated with Poly I:C (30 ng/ml) for 7 days in differentiation medium I containing DMEM/FBS and 7.5% knockout serum replacement (KSR). Following 7 days, the medium was changed to differentiation medium II containing DMEM/FBS and 10% KSR containing 20 ng/ml BMP4, 50 ng/ml VEGF and 20 ng/ml bFGF. After another 7 days, the medium was replaced with endothelial medium (EGM2) containing 50 ng/ml VEGF, 20 ng/ml bFGF and 0.1 mM 8-Br-cAMP for another 14 days. The medium was changed every 2-3 days. Cells were then FAC sorted with CD31 or VE-cadherin and then expanded in EGM2 medium containing SB431542, a specific TGFβ receptor inhibitor. (B) Fluorescent activated cell sorting (FACs) plot of data obtained using CD31+ antibody to quantitate iECs. (left panel)—vehicle control; (middle panel)—Poly I:C and (right panel)—enrichment of iECs with CD31 antibody. (C-D) Real-Time RT-PCR and immunofluorescent staining of iECs for endothelial markers CD31, CD144, eNOS, and von Willebrand factor. (E-F) iECs take up acetylated LDL and forms capillary-like networks on matrigel.

FIG. 22: Improvement of blood perfusion and capillary density in ischemic hind limbs by iEC transplantation. (A) Representative images of laser Doppler perfusion imaging. (B) Summarized data of perfusion ratio (value of the ischemic limb divided by that of non-ischemic limb) at day 0 and 7 post-treatment. (C) Immunofluorescence CD31 staining of ischemic tissues from mice treated with iECs or vehicle. (D) Quantification of total capillary density in the ischemic limbs. (E) Hind limb ischemia score obtained by blinded observers.

FIG. 23: TLR3 signaling enables efficient transdifferentiation of human fibroblasts to functional endothelial cells: (A) Protocol for direct reprogramming of human BJ fibroblasts to endothelial cells in TLR3 knockdown cells (described above). (B) Fluorescent activated cell sorting (FACs) plot of data obtained using CD144+ antibody to quantitate iECs (left panel)—scramble cells treated with vehicle control; (middle panel)—scramble cells treated with Poly I:C and (right panel)—TLR3-KD cells treated with Poly I:C. (C-D) iECs derived from TLR3-KD cells have reduced capacity to uptake acetylated LDL and fails to form capillary-like networks on matrigel.

### DETAILED DESCRIPTION OF THE EMBODIMENTS

The reprogramming of a somatic cells to an induced pluripotent stem cell or transdifferentiated cell with non-integrating programming factors is shown to be greatly accelerated by activation of innate immune responses in the somatic cell. Methods of activating innate immunity include activation of toll-like receptors, e.g. TLR3.

### DEFINITIONS

It is to be understood that this invention is not limited to the particular methodology, protocols, cell lines, animal species or genera, and reagents described, as such may vary. It is also to be understood that the terminology used herein is for the purpose of describing particular embodiments only, and is not intended to limit the scope of the present invention which will be limited only by the appended claims.

As used herein the singular forms “a”, “and”, and “the” include plural referents unless the context clearly dictates otherwise. Thus, for example, reference to “a cell” includes a plurality of such cells and reference to “the protein” includes reference to one or more proteins and equivalents thereof known to those skilled in the art, and so forth. All technical and scientific terms used herein have the same meaning as commonly understood to one of ordinary skill in the art to which this invention belongs unless clearly indicated otherwise.

Innate Immunity.

The innate immune system is a primitive cellular response that provides for a defense of cells against pathogen antigens. Recognition of these antigens by the innate immune system may result in an inflammatory response characterized by the production of cytokines such as TNF, IL-1, IL-6, and IL-8; as well as gene activation of ICAM-1 and E-selectin, among others.

The broad classes of pathogens, e.g. viruses, bacteria, and fungi, may constitutively express a set of class-specific, mutation-resistant molecules called pathogen-associated molecular patterns (PAMPs). These microbial molecular markers may be composed of proteins, carbohydrates, lipids, nucleic acids and/or combinations thereof, and may be located internally or externally. Examples include the endotoxin lipopolysaccharide (LPS), single or double-stranded RNA, and the like.

Typically PAMP receptors (PRRs) are nonclonal, i.e. expressed on all cells of a given type, and germ-line encoded, or independent of immunologic memory. Once bound, PRRs tend to cluster, recruit other extracellular and intracellular proteins to the complex, and initiate signaling cascades that ultimately impact transcription. Further, PRRs are involved in activation of complement, coagulation, phagocytosis, inflammation, and apoptosis functions in response to pathogen detection. There are several types of PRRs including complement, glucan, mannose, scavenger, and toll-like receptors, each with specific PAMP ligands, expression patterns, signaling pathways, and anti-pathogen responses.

The Toll-like receptors are type I transmembrane (TM) PRRs that possess varying numbers of extracellular N-terminal leucine-rich repeat (LRR) motifs, followed by a cysteine-rich region, a TM domain, and an intracellular Toll/IL-1 R (TIR) motif. The LLR domain is important for ligand binding and associated signaling and is a common feature of PRRs. The TIR domain is important in protein-protein interactions and is typically associated with innate immunity. The TIR domain also unites a larger IL-1 R/TLR superfamily that is composed of three subgroups. The human TLR family is composed of at least 10 members, TLR1 through 10. Each TLR is specific in its expression patterns and PAMP sensitivities.

Toll-like receptor 3 (TLR3) recognizes double-stranded RNA (dsRNA) and mimetics thereof, a molecular pattern associated with viral infection. It maps to chromosome 4q35 and its sequence encodes a putative 904 aa protein with 24 N-terminal LRRs and a calculated molecular weight of 97 kDa. TLR3 is most closely related to TLR5, TLR7, and TLR8, each with 26% overall aa sequence identity. TLR3 mRNA is elevated after exposure to Gram-negative bacteria and to an even greater extent in response to Gram-positive bacteria.

TLR3 specifically recognizes double-stranded RNA (dsRNA) and induces multiple intracellular events responsible for innate antiviral immunity against a number of viral infections. The predicted 904-amino acid TLR3 protein contains the characteristic Toll motifs: an extracellular leucine-rich repeat (LRR) domain and a cytoplasmic interleukin-1 receptor-like region.

Exposure to double-stranded RNA (dsRNA) or polyinosine-polycytidylic acid (poly(I:C)), a synthetic dsRNA analog, induces the production of interferon α and β and by signaling through TLR3 activates NFκB. IRF3 is specifically induced by stimulation of TLR3 or TLR4, which mediates a specific gene program responsible for innate antiviral responses. TRIF is necessary for TLR3-dependent activation of NFKB. It serves as an adaptor protein linking RIP1 and TLR3 to mediate TLR3-induced NFKB activation.

RIG-1 (retinoic acid-inducible gene 1) is a RIG-1-like receptor dsRNA helicase enzyme that is encoded (in humans) by the DDX58 gene. RIG-I is part of the RIG-1-like receptor (RLR) family, which also includes MDA5 and LGP2, and functions as a pattern recognition receptor that is a sensor for viruses. RIG-I typically recognizes short (<4000 nt) 5′ triphosphate dsRNA. RIG-I and MDA5 are involved in activating MAVS and triggering an antiviral response. The human RIG1 gene may be accessed at Genbank NM_{—}014314.3 and the protein at Genbank NP_{—}055129.2.

Toll-like receptor 4 is a protein that in humans is encoded by the TLR4 gene. It detects lipopolysaccharide from Gram-negative bacteria and is thus important in the activation of the innate immune system. This receptor is most abundantly expressed in placenta, and in myelomonocytic subpopulation of the leukocytes. The human TLR4 gene may be accessed at Genbank NM_{—}003266.3 and the protein accessed at Genbank NP_{—}003257.1.

Activation of TLR4 leads to downstream release of inflammatory modulators including TNF-α and Interleukin-1. Agonists include morphine, oxycodone, fentanyl, methadone, lipopolysaccharides (LPS), carbamazepine, oxcarbazepine, etc.

TLR Agonist.

TLR agonists activate TLRs, including without limitation TLR3, TLR4, and RIG1. Examples of TLR agonists include pathogen-associated molecular patterns (PAMPs) and mimetics thereof. These microbial molecular markers may be composed of proteins, carbohydrates, lipids, nucleic acids and/or combinations thereof, and may be located internally or externally, as known in the art. Examples include LPS, zymosan, peptidoglycans, flagellin, synthetic TLR2 agonist Pam3cys, Pam3CSK4, MALP-2, Imiquimod, CpG ODN, and the like.

TLR3 agonists include double-stranded RNA; Poly(I:C), Poly(A.U), etc., where such nucleic acids usually have a size of at least about 10 bp, at least about 20 bp, at least about 50 bp and may have a high molecular weight of from about 1 to about 20 kb, usually not more than about 50 to 100 kb. Alternative TLR3 agonists may directly bind to the protein, e.g. antibodies or small molecules that selectively bind to and activate TLR3. Other TLR3 agonists include retroviruses, e.g. a retrovirus engineered to lack the ability to integrate into the genome.

The dose of agonist that is effective in the methods of the invention is a dose that increases the efficiency of reprogramming of a cell or cell population, relative to the same population in the absence of the TLR agonist. The term “reprogramming” as used here means nuclear reprogramming of a somatic cell to a pluripotential cell (eg. a fibroblast to an induced pluripotential cell) or nuclear reprogramming of a somatic cell to a substantially different somatic cell (eg. a fibroblast to an endothelial cell), in vitro or in vivo. The latter process is also known as transdifferentiation.

Conveniently, a marker of TLR activation may be assessed for the determination of suitable doses, including the activation of NFκB in the somatic cells of interest for reprogramming, production of interferons α and β, and the like. For example, where the TLR agonist of poly I:C or an analog thereof, an effective dose may be at least about 10 ng/ml, at least about 50 ng/ml, at least about 100 ng/ml, at least about 250 ng/ml, at least about 500 ng/ml. An optimized concentration of poly I:C in culture medium is at least 10 ng/ml and not more than 3000 ng/ml, including a range from 20 ng/ml to 300 ng/ml, and particularly from 25 ng/ml to 150 ng/ml, for example around 30 ng/ml. The dose of a TLR agonist other than poly I:C may be calculated based on the provision of activity equivalent to the optimized poly I:C dose.

By “pluripotency” and pluripotent stem cells it is meant that such cells have the ability to differentiate into all types of cells in an organism. The term “induced pluripotent stem cell” encompasses pluripotent cells, that, like embryonic stem (ES) cells, can be cultured over a long period of time while maintaining the ability to differentiate into all types of cells in an organism, but that, unlike ES cells (which are derived from the inner cell mass of blastocysts), are derived from differentiated somatic cells, that is, cells that had a narrower, more defined potential and that in the absence of experimental manipulation could not give rise to all types of cells in the organism. By “having the potential to become iPS cells” it is meant that the differentiated somatic cells can be induced to become, i.e. can be reprogrammed to become, iPS cells. In other words, the somatic cell can be induced to redifferentiate so as to establish cells having the morphological characteristics, growth ability and pluripotency of pluripotent cells. iPS cells have an hESC-like morphology, growing as flat colonies with large nucleo-cytoplasmic ratios, defined borders and prominent nuclei. In addition, iPS cells express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. In addition, the iPS cells are capable of forming teratomas. In addition, they are capable of forming or contributing to ectoderm, mesoderm, or endoderm tissues in a living organism.

The terms “primary cells”, “primary cell lines”, and “primary cultures” are used interchangeably herein to refer to cells and cell cultures that have been derived from a subject and allowed to grow in vitro for a limited number of passages, i.e. splittings, of the culture. For example primary cultures are cultures that may have been passaged 0 times, 1 time, 2 times, 4 times, 5 times, 10 times, or 15 times, but not enough times go through the crisis stage.

Starting Cell Population.

As used herein, a “starting cell population”, or “initial cell population” refers to a somatic cell, usually a primary, or non-transformed, somatic cell, which undergoes nuclear reprogramming by the methods of the invention. The starting cell population may be of any mammalian species, but particularly including human cells. Sources of starting cell populations include individuals desirous of cellular therapy, individuals having a genetic defect of interest for study, and the like.

In some embodiments, human cells obtained from a subject for the purpose of nuclear reprogramming may be chosen from any human cell type, including fibroblast cells, adipose tissue cells, mesenchymal cells, bone marrow cells, stomach cells, liver cells, epithelial cells, nasal epithelial cells, mucosal epithelial cells, follicular cells, connective tissue cells, muscle cells, bone cells, cartilage cells, gastrointestinal cells, splenic cells, kidney cells, lung cells, testicular cells, nervous tissue cells, etc. In some embodiments, the human cell type is a fibroblast, which may be conveniently obtained from a subject by a punch biopsy. In certain embodiments, the cells are obtained from subjects known or suspected to have a copy number variation (CNV) or mutation of the gene of interest. In other embodiments, the cells are from a patient presenting with idiopathic/sporadic form of the disease. In yet other embodiments, the cells are from a non-human subject. The cells are then reprogrammed, and may be transdifferentiated to adopt a specific cell fate, such as endodermal cells, neuronal cells, for example dopaminergic, cholinergic, serotonergic, GABAergic, or glutamatergic neuronal cell; pancreatic cells, e.g. islet cells, muscle cells including without limitation cardiomyocytes, hematopoietic cells, and the like.

The term “efficiency of reprogramming” may be used to refer to the ability of a cells to give rise to iPS cell colonies when contacted with reprogramming factors. Somatic cells that demonstrate an enhanced efficiency of reprogramming to pluripotentiality will demonstrate an enhanced ability to give rise to iPS cells when contacted with reprogramming factors relative to a control. The term “efficiency of reprogramming” may also refer to the ability of somatic cells to be reprogrammed to a substantially different somatic cell type, a process known as transdifferentiation. The efficiency of reprogramming with the methods of the invention vary with the particular combination of somatic cells, method of introducing reprogramming factors, and method of culture following induction of reprogramming.

Reprogramming factors, as used herein, refers to one or a cocktail of biologically active polypeptides or small molecules that act on a cell to alter transcription, and which upon expression, reprogram a somatic cell a different cell type, or to multipotency or to pluripotency. For the purposes of the present invention, it is desirable that the reprogramming factors be non-integrating, i.e. provided to the recipient somatic cell in a form that does not result in integration of exogenous DNA into the genome of the recipient cell. As such, agents other than nucleic acids, e.g. proteins and small molecules are often preferred.

For the purposes of the present invention, reprogramming factors are usually fused to a permeant domain to allow entry of the polypeptide across a cell membrane and across the nuclear membrane. Reprogramming factors may be of any suitable mammalian species, e.g. human, murine, porcine, equine, canine, ovine, feline, simian, etc. Human polypeptides are of particular interest.

In some embodiments the reprogramming factor is a transcription factor, including without limitation, Oct3/4; Sox2; Klf4; c-Myc; and Nanog. Also of interest as a reprogramming factor is Lin28, which is an mRNA-binding protein thought to influence the translation or stability of specific mRNAs during differentiation.

Reprogramming factors of interest also include factors useful in transdifferentiation, where a somatic cell is reprogrammed to a different somatic cell. For the purpose of transdifferentiation of one somatic cell to another, substantially different, somatic cell type, a different set of reprogramming factors find use. For example, to transdifferentiate a fibroblast to a cardiomyocyte, one might use cell permeant peptides Gata4, Mef2c and Tbx5 (Leda et al used viral vectors to convert fibroblasts to cardiomyocytes; Cell, Volume 142, Issue 3, 375-386, 6 Aug. 2010, herein specifically incorporated by reference.)

The reprogramming factors may be provided as a composition of isolated polypeptide, i.e. in a cell-free form, which is biologically active. Biological activity may be determined by specific DNA binding assays, as described in the Examples; or by determining the effectiveness of the factor in altering cellular transcription. A composition of the invention may provide one or more biologically active reprogramming factors. The composition may comprise at least about 50 μg/ml soluble reprogramming factor, at least about 100 μg/ml; at least about 150 μg/ml, at least about 200 μg/ml, at least about 250 μg/ml, at least about 300 μg/ml, or more.

A Klf4 polypeptide is a polypeptide comprising the amino acid sequence that is at least 70% identical to the amino acid sequence of human Klf4, i.e., Kruppel-Like Factor 4 the sequence of which may be found at GenBank Accession Nos. NP_{—}004226 and NM_{—}004235. Klf4 polypeptides, e.g. those that are at least 70%, 75%, 80%, 85%, 90%, 91%, 92%, 95%, 97%, 99%, or 100% identical to the sequence provided in GenBank Accession No. NM_{—}004235, and the nucleic acids that encode them find use as a reprogramming factor in the present invention.

A c-Myc polypeptide is a polypeptide comprising an amino acid sequence that is at least 70% identical to the amino acid sequence of human c-Myc, i.e., myelocytomatosis viral oncogene homolog, the sequence of which may be found at GenBank Accession Nos. NP_{—}002458 and NM_{—}002467. c-Myc polypeptides, e.g. those that are at least 70%, 75%, 80%, 85%, 90%, 91%, 92%, 95%, 97%, 99%, or 100% identical to the sequence provided in GenBank Accession No. NM_{—}002467, and the nucleic acids that encode them find use as a reprogramming factor in the present invention.

A Nanog polypeptide is a polypeptide comprising an amino acid sequence that is at least 70% identical to the amino acid sequence of human Nanog, i.e., Nanog homeobox, the sequence of which may be found at GenBank Accession Nos. NP_{—}079141 and NM_{—}024865. Nanog polypeptides, e.g. those that are at least 70%, 75%, 80%, 85%, 90%, 91%, 92%, 95%, 97%, 99%, or 100% identical to the sequence provided in GenBank Accession No. NM_{—}024865, and the nucleic acids that encode them find use as a reprogramming factor in the present invention.

A Lin-28 polypeptide is a polypeptide comprising an amino acid sequence that is at least 70% identical to the amino acid sequence of human Lin-28, i.e., Lin-28 homolog of C. elegans, the sequence of which may be found at GenBank Accession Nos. NP_{—}078950 and NM_{—}024674. Lin-28 polypeptides, e.g. those that are at least 70%, 75%, 80%, 85%, 90%, 91%, 92%, 95%, 97%, 99%, or 100% identical to the sequence provided in GenBank Accession No. NM_{—}024674, and the nucleic acids that encode them find use as a reprogramming factor in the present invention.

An Oct3/4 polypeptide is a polypeptide comprising an amino acid sequence that is at least 70% identical to the amino acid sequence of human Oct3/4, also known as Homo sapiens POU class 5 homeobox 1 (POU5F1) the sequence of which may be found at GenBank Accession Nos. NP_{—}002692 and NM_{—}002701. Oct3/4 polypeptides, e.g. those that are at least 70%, 75%, 80%, 85%, 90%, 91%, 92%, 95%, 97%, 99%, or 100% identical to the sequence provided in GenBank Accession No. NM_{—}002701, and the nucleic acids that encode them find use as a reprogramming factor in the present invention.

A Sox2 polypeptide is a polypeptide comprising the amino acid sequence at least 70% identical to the amino acid sequence of human Sox2, i.e., sex-determining region Y-box 2 protein, the sequence of which may be found at GenBank Accession Nos. NP_{—}003097 and NM_{—}003106. Sox2 polypeptides, e.g. those that are at least 70%, 75%, 80%, 85%, 90%, 91%, 92%, 95%, 97%, 99%, or 100% identical to the sequence provided in GenBank Accession No. NM_{—}003106, and the nucleic acids that encode them find use as a reprogramming factor in the present invention.

Small molecules, including without limitation valproic acid, hydroxamic acid, trichostatin A, suberoylanilide hydroxamic acid, BIX-01294 and BayK8644 have been described as useful in reprogramming cells (see Shi et al. (2008) Cell Stem Cell 6; 3(5):568-574 and Huangfu et al. (2008) Nature Biotechnology 26:795-797, each herein specifically incorporated by reference).

The terms “treatment”, “treating”, “treat” and the like are used herein to generally refer to obtaining a desired pharmacologic and/or physiologic effect. The effect may be prophylactic in terms of completely or partially preventing a disease or symptom thereof and/or may be therapeutic in terms of a partial or complete stabilization or cure for a disease and/or adverse effect attributable to the disease. “Treatment” as used herein covers any treatment of a disease in a mammal, particularly a human, and includes: (a) preventing the disease or symptom from occurring in a subject which may be predisposed to the disease or symptom but has not yet been diagnosed as having it; (b) inhibiting the disease symptom, i.e., arresting its development; or (c) relieving the disease symptom, i.e., causing regression of the disease or symptom.

The terms “individual,” “subject,” “host,” and “patient,” are used interchangeably herein and refer to any mammalian subject for whom diagnosis, treatment, or therapy is desired, particularly humans.

Permeant Domain.

A number of permeant domains are known in the art and may be used in the present invention, including peptides, peptidomimetics, and non-peptide carriers. In one embodiment, the permeant peptide is derived from the third alpha helix of Drosophila melanogaster transcription factor Antennapaedia, referred to as penetratin, which comprises the amino acid sequence RQIKIWFQNRRMKWKK. In another embodiment, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

### Methods of Inducing Pluripotency In Vitro

A starting population of somatic cells are contacted with reprogramming factors, as defined above, in a combination and quantity sufficient to reprogram the cell to pluripotency prior to, concurrent with or following activation of the somatic cell with an effective dose of an activator of innate immunity, e.g. a TLR agonist. In one embodiment of the invention, the TLR is TLR3. In some embodiments the TLR agonist is a double-stranded RNA or analog thereof. Reprogramming factors may be provided to the somatic cells individually or as a single composition, that is, as a premixed composition, of reprogramming factors.

In some embodiments, the starting population of cells is contacted with an effective dose of a TLR agonist, e.g. LPS, dsRNA, etc., in a dose that is functionally equivalent to a dose of from 5 ng/ml to 3000 ng/ml poly I:C, and maintained in culture in the presence of such an agonist from a period of time from about 4 to about 18 days, e.g. from about 5 to about 10 days, and may be around 6 to 8 days.

The reprogramming factors may be added to the subject cells simultaneously or sequentially at different times, and may be added in combination with the activator of innate immunity. In some embodiments, a set of at least three purified reprogramming factor is added, e.g., an Oct3/4 polypeptide, a Sox2 polypeptide, and a Klf4, c-myc, nanog or lin28 polypeptide. In some embodiments, a set of four purified reprogramming factors is provided to the cells e.g., an Oct3/4 polypeptide, a Sox2 polypeptide, a Klf4 polypeptide and a c-Myc polypeptide; or an Oct3/4 polypeptide, a Sox2 polypeptide, a lin28 polypeptide and a nanog polypeptide.

Methods for introducing the reprogramming factors to somatic cells include providing a cell with purified protein factors. Typically, a reprogramming factor polypeptide will comprise the polypeptide sequences of the reprogramming factor fused to a polypeptide permeant domain. A number of permeant domains are known in the art and may be used in the nuclear acting, non-integrating polypeptides of the present invention, including peptides, peptidomimetics, and non-peptide carriers. For example, a permeant peptide may be derived from the third alpha helix of Drosophila melanogaster transcription factor Antennapaedia, referred to as penetratin, which comprises the amino acid sequence RQIKIWFQNRRMKWKK. As another example, the permeant peptide comprises the HIV-1 tat basic region amino acid sequence, which may include, for example, amino acids 49-57 of naturally-occurring tat protein. Other permeant domains include poly-arginine motifs, for example, the region of amino acids 34-56 of HIV-1 rev protein, nona-arginine, octa-arginine, and the like. (See, for example, Futaki et al. (2003) Curr Protein Pept Sci. 2003 April; 4(2): 87-96; and Wender et al. (2000) Proc. Natl. Acad. Sci. U.S.A 2000 Nov. 21; 97(24):13003-8; published U.S. Patent applications 20030220334; 20030083256; 20030032593; and 20030022831, herein specifically incorporated by reference for the teachings of translocation peptides and peptoids). The nona-arginine (R9) sequence is one of the more efficient PTDs that have been characterized (Wender et al. 2000; Uemura et al. 2002).

In such embodiments, cells are incubated in the presence of a purified reprogramming factor polypeptide for about 30 minutes to about 72 hours, e.g., 2 hours, 4 hours, 8 hours, 12 hours, 18 hours, 24 hours 36 hours, 48 hours, 60 hours, 72 hours, or any other period from about 30 minutes to about 72 hours. Typically, the reprogramming factors are provided to the subject cells four times, and the cells are allowed to incubate with the reprogramming factors for 48 hours, after which time the media is replaced with fresh media and the cells are cultured further (See, for example, Zhou et al. (2009) Cell Stem Cells 4(5); 381-384). The reprogramming factors may be provided to the subject cells for about one to about 4 weeks, e.g. from about two to about 3 weeks.

The dose of reprogramming factors will vary with the nature of the cells, the factors, the culture conditions, etc. In some embodiments the dose will be from about 1 nM to about 1 μM for each factor, more usually from about 10 nM to about 500 nM, or around about 100 to 200 nM. Conveniently the cells are initially exposed to a TLR agonist during exposure to the reprogramming actors for at least about 1 day, at least about 2 days, at least about 4 days, at least about 6 days or one week, and may be exposed for the entire reprogramming process, or less. The dose will depend on the specific agonist, but may be from about 1 ng/ml to about 1 μg/ml, from about 10 ng/ml to about 500 ng/ml. Two 16-24 hour incubations with the recombination factors may follow each provision, after which the media is replaced with fresh media and the cells are cultured further.

In some embodiments, a vector that does not integrate into the somatic cell genome is used. Many vectors useful for transferring exogenous genes into target mammalian cells are available. The vectors may be maintained episomally, e.g. as plasmids, virus-derived vectors such cytomegalovirus, adenovirus, etc. Vectors used for providing reprogramming factors to the subject cells as nucleic acids will typically comprise suitable promoters for driving the expression, that is, transcriptional activation, of the reprogramming factor nucleic acids. This may include ubiquitously acting promoters, for example, the CMV-β-actin promoter, or inducible promoters, such as promoters that are active in particular cell populations or that respond to the presence of drugs such as tetracycline. By transcriptional activation, it is intended that transcription will be increased above basal levels in the target cell by at least about 10 fold, by at least about 100 fold, more usually by at least about 1000 fold.

Following introduction of reprogramming factors, the somatic cells may be maintained in a conventional culture medium comprising feeder layer cells, or may be cultured in the absence of feeder layers, i.e. lacking somatic cells other than those being induced to pluripotency. Feeder layer free cultures may utilize a protein coated surface, e.g. matrigel, etc.

iPS cells induced to become such by the methods of the invention have an hESC-like morphology, growing as flat colonies with large nucleo-cytoplasmic ratios, defined borders and prominent nuclei. In addition, the iPS cells may express one or more key pluripotency markers known by one of ordinary skill in the art, including but not limited to Alkaline Phosphatase, SSEA3, SSEA4, Sox2, Oct3/4, Nanog, TRA160, TRA181, TDGF 1, Dnmt3b, FoxD3, GDF3, Cyp26a1, TERT, and zfp42. In addition, the iPS cells are capable of forming teratomas. In addition, they are capable of forming or contributing to ectoderm, mesoderm, or endoderm tissues in a living organism.

Genes may be introduced into the somatic cells or the iPS cells derived therefrom for a variety of purposes, e.g. to replace genes having a loss of function mutation, provide marker genes, etc. Alternatively, vectors are introduced that express antisense mRNA or ribozymes, thereby blocking expression of an undesired gene. Other methods of gene therapy are the introduction of drug resistance genes to enable normal progenitor cells to have an advantage and be subject to selective pressure, for example the multiple drug resistance gene (MDR), or anti-apoptosis genes, such as bcl-2. Various techniques known in the art may be used to introduce nucleic acids into the target cells, e.g. electroporation, calcium precipitated DNA, fusion, transfection, lipofection, infection and the like, as discussed above. The particular manner in which the DNA is introduced is not critical to the practice of the invention.

The iPS cells produced by the above methods may be used for reconstituting or supplementing differentiating or differentiated cells in a recipient. The induced cells may be differentiated into cell-types of various lineages. Examples of differentiated cells include any differentiated cells from ectodermal (e.g., neurons and fibroblasts), mesodermal (e.g., cardiomyocytes), or endodermal (e.g., pancreatic cells) lineages. The differentiated cells may be one or more: pancreatic beta cells, neural stem cells, neurons (e.g., dopaminergic neurons), oligodendrocytes, oligodendrocyte progenitor cells, hepatocytes, hepatic stem cells, astrocytes, myocytes, hematopoietic cells, or cardiomyocytes.

There are numerous methods of differentiating the induced cells into a more specialized cell type. Methods of differentiating induced cells may be similar to those used to differentiate stem cells, particularly ES cells, MSCs, MAPCs, MIAMI, hematopoietic stem cells (HSCs). In some cases, the differentiation occurs ex vivo; in some cases the differentiation occurs in vivo.

The induced cells, or cells differentiated from the induced cells, may be used as a therapy to treat disease (e.g., a genetic defect). The therapy may be directed at treating the cause of the disease; or alternatively, the therapy may be to treat the effects of the disease or condition. The induced cells may be transferred to, or close to, an injured site in a subject; or the cells can be introduced to the subject in a manner allowing the cells to migrate, or home, to the injured site. The transferred cells may advantageously replace the damaged or injured cells and allow improvement in the overall condition of the subject. In some instances, the transferred cells may stimulate tissue regeneration or repair.

The transferred cells may be cells differentiated from induced cells. The transferred cells also may be multipotent stem cells differentiated from the induced cells. In some cases, the transferred cells may be induced cells that have not been differentiated.

The number of administrations of treatment to a subject may vary. Introducing the induced and/or differentiated cells into the subject may be a one-time event; but in certain situations, such treatment may elicit improvement for a limited period of time and require an on-going series of repeated treatments. In other situations, multiple administrations of the cells may be required before an effect is observed. The exact protocols depend upon the disease or condition, the stage of the disease and parameters of the individual subject being treated.

The cells may be introduced to the subject via any of the following routes: parenteral, intravenous, intraarterial, intramuscular, subcutaneous, transdermal, intratracheal, intraperitoneal, or into spinal fluid.

The iPS cells may be administered in any physiologically acceptable medium. They may be provided alone or with a suitable substrate or matrix, e.g. to support their growth and/or organization in the tissue to which they are being transplanted. Usually, at least 1×10⁵ cells will be administered, preferably 1×10⁶ or more. The cells may be introduced by injection, catheter, or the like. The cells may be frozen at liquid nitrogen temperatures and stored for long periods of time, being capable of use on thawing. If frozen, the cells will usually be stored in a 10% DMSO, 50% FCS, 40% RPMI 1640 medium. Once thawed, the cells may be expanded by use of growth factors and/or stromal cells associated with progenitor cell proliferation and differentiation.

Kits may be provided, where the kit will comprise an effective dose of a TLR agonist. In some embodiments the TLR agonist is a TLR3 agonist, e.g. a double stranded RNA or analog thereof. The kit may further comprise one or more reprogramming factors, e.g. in the form of proteins fused to a permeant domain.

### Methods of Inducing Transdifferentiation In Vitro or In Vivo

Transdifferentiation, as defined above, is the nuclear reprogramming of a somatic cell to a substantially different somatic cell, for example a somatic cell of a different lineage. Examples of transdifferentiation include, without limitation: fibroblast→myocyte; fibroblast→endothelial cell; fibroblast→neural cell; fibroblast→islet cell; fibroblast→hematopoietic cell; etc.; adipose tissue cell to any one of myocytes, endothelial cell, neural cell, hematopoietic cell, islet cell, etc.; and the like. Cells suitable as a starting populations have been defined above. This methodology can provide for consistency and practical application in regenerative medicine.

For the purpose of transdifferentiation, a different set of factors and media specific to the derived cell phenotype, will be used. Typically, the differentiating factors will be provided to the subject cell after the cell has been exposed to Poly I:C for a period of time sufficient to induce innate immunity. In some embodiments the cell is exposed to an activator of innate immunity in the absence of differentiating factors. In some embodiments, the starting population of cells is contacted with an effective dose of a TLR agonist, e.g. LPS, dsRNA, etc., in a dose that is functionally equivalent to a dose of from 5 ng/ml to 3000 ng/ml poly I:C, and maintained in culture in the presence of such an agonist from a period of time from about 4 to about 18 days, e.g. from about 5 to about 10 days, and may be around 6 to 8 days. Following induction of innate immunity by this process, the cells is exposed to one or a cocktail of differentiating factors.

For example, following TLR agonist treatment with an effective dose for about one week, cells are transdifferentiated by exposing them to differentiating factors for an additional one to four weeks. The medium may be replaced with fresh medium supplemented with growth factors specific for the cell being derived. The appropriate concentration of the factors required is determined by conducting a dose-response curve. Similarly, the transdifferentiated cells are characterized with a series of standard secondary assays including gene expression, morphological and functional analysis. In many embodiments, culture protocols used for differentiation of a somatic cell type from a pluripotent cell population, e.g. ES cells, iPS cells, etc. can be applied to transdifferentiation. That is, a cell that has been exposed to a TLR agonist in culture for a period of time sufficient to induce innate immunity can then be exposed to a conventional set of factors for lineage specific differentiation.

The cells may be differentiated into cell-types of various lineages. Examples of transdifferentiated cells include any differentiated cells from ectodermal (e.g., neurons and fibroblasts), mesodermal (e.g., cardiomyocytes), or endodermal (e.g., endodermal cells, pancreatic cells) lineages. The transdifferentiated cells may be one or more: pancreatic beta cells, neural stem cells, neurons (e.g., dopaminergic neurons), oligodendrocytes, oligodendrocyte progenitor cells, hepatocytes, hepatic stem cells, astrocytes, myocytes, hematopoietic cells, endodermal cells, or cardiomyocytes, etc.

The transdifferentiated cells may be terminally differentiated cells, or they may be capable of giving rise to cells of a specific lineage. For example, cells can be differentiated into a variety of multipotent cell types, e.g., neural stem cells, cardiac stem cells, or hepatic stem cells. The stem cells may then be further differentiated into new cell types, e.g., neural stem cells may be differentiated into neurons; cardiac stem cells may be differentiated into cardiomyocytes; and hepatic stem cells may be differentiated into hepatocytes.

There are numerous methods of differentiating the induced cells into a more specialized cell type. Methods of differentiating induced cells may be similar to those used to differentiate stem cells, particularly ES cells, MSCs, MAPCs, MIAMI, hematopoietic stem cells (HSCs). In some cases, the differentiation occurs ex vivo; in some cases the differentiation occurs in vivo.

In some embodiments the TLR agonist-treated cells are differentiated into endothelial cells, for example with the protocol set forth in Example 2 herein. Following treatment with poly I:C, the cells are cultured in medium comprising an effective dose of BMP4, VEGF and bFGF. After another 5-10 days, the medium was replaced with endothelial medium comprising an effective dose of VEGF, bFGF and 8-Br-cAMP for another 10-20 days. The resulting endothelial cells may be used as is, or can be further expanded in culture, e.g. in the presence of medium comprising an effective dose of a TGF receptor inhibitor.

Any known method of generating neural stem cells from ES cells may be used to generate neural stem cells from TLR agonist-treated cells. See, e.g., Reubinoff et al., (2001), Nat, Biotechnol., 19(12): 1134-40. For example, neural stem cells may be generated by culturing the TLR agonist-treated cells as floating aggregates in the presence of noggin, or other bone morphogenetic protein antagonist, see e.g., Itsykson et al., (2005), Mol Cell Neurosci., 30(1):24-36. In another example, neural stem cells may be generated by culturing the TLR agonist-treated cells in suspension to form aggregates in the presence of growth factors, e.g., FGF-2, Zhang et al., (2001), Nat. Biotech., (19): 1129-1133. In some cases, the aggregates are cultured in serum-free medium containing FGF-2. In another example, the TLR agonist-treated cells are co-cultured with a mouse stromal cell line, e.g., PA6 in the presence of serum-free medium comprising FGF-2. In yet another example, the TLR agonist-treated cells are directly transferred to serum-free medium containing FGF-2 to directly induce differentiation.

Neural stems derived from the TLR agonist-treated cells may be differentiated into neurons, oligodendrocytes, or astrocytes. Often, the conditions used to generate neural stem cells can also be used to generate neurons, oligodendrocytes, or astrocytes.

Dopaminergic neurons play a central role in Parkinson's Disease and other neurodegenerative diseases and are thus of particular interest. In order to promote differentiation into dopaminergic neurons, TLR agonist-treated cells may be co-cultured with a PA6 mouse stromal cell line under serum-free conditions, see, e.g., Kawasaki et al., (2000) Neuron, 28(1):3140. Other methods have also been described, see, e.g., Pomp et al., (2005), Stem Cells 23(7):923-30; U.S. Pat. No. 6,395,546, e.g., Lee et al., (2000), Nature Biotechnol., 18:675-679.

Oligodendrocytes may also be generated from the induced cells. Differentiation of the induced cells into oligodendrocytes may be accomplished by known methods for differentiating ES cells or neural stem cells into oligodendrocytes. For example, oligodendrocytes may be generated by co-culturing induced cells or neural stem cells with stromal cells, e.g., Hermann et al. (2004), J Cell Sci. 117(Pt 19):4411-22. In another example, oligodendrocytes may be generated by culturing the induced cells or neural stem cells in the presence of a fusion protein, in which the Interleukin (IL)-6 receptor, or derivative, is linked to the IL-6 cytokine, or derivative thereof. Oligodendrocytes can also be generated from the induced cells by other methods known in the art, see, e.g. Kang et al., (2007) Stem Cells 25, 419-424.

Astrocytes may also be produced from the TLR agonist-treated cells. Astrocytes may be generated by culturing TLR agonist-treated cells or neural stem cells in the presence of neurogenic medium with bFGF and EGF, see e.g., Brustle et al., (1999), Science, 285:754-756.

TLR agonist-treated cells may be differentiated into pancreatic beta cells by methods known in the art, e.g., Lumelsky et al., (2001) Science, 292:1389-1394; Assady et al., (2001), Diabetes, 50:1691-1697; D'Amour et al., (2006), Nat. Biotechnol., 24:1392-1401; D'Amour et al., (2005), Nat. Biotechnol. 23:1534-1541. The method may comprise culturing the TLR agonist-treated cells in serum-free medium supplemented with Activin A, followed by culturing in the presence of serum-free medium supplemented with all-trans retinoic acid, followed by culturing in the presence of serum-free medium supplemented with bFGF and nicotinamide, e.g., Jiang et al., (2007), Cell Res., 4:333-444. In other examples, the method comprises culturing the TLR agonist-treated cells in the presence of serum-free medium, activin A, and Wnt protein from about 0.5 to about 6 days, e.g., about 0.5, 1, 2, 3, 4, 5, 6, days; followed by culturing in the presence of from about 0.1% to about 2%, e.g., 0.2%, FBS and activin A from about 1 to about 4 days, e.g., about 1, 2, 3, or 4 days; followed by culturing in the presence of 2% FBS, FGF-10, and KAAD-cyclopamine (keto-N-aminoethylaminocaproyl dihydro cinnamoylcyclopamine) and retinoic acid from about 1 to about 5 days, e.g., 1, 2, 3, 4, or 5 days; followed by culturing with 1% B27, gamma secretase inhibitor and extendin-4 from about 1 to about 4 days, e.g., 1, 2, 3, or 4 days; and finally culturing in the presence of 1% B27, extendin-4, IGF-1, and HGF for from about 1 to about 4 days, e.g., 1, 2, 3, or 4 days.

Hepatic cells or hepatic stem cells may be differentiated from the TLR agonist-treated cells. For example, culturing the TLR agonist-treated cells in the presence of sodium butyrate may generate hepatocytes, see e.g., Rambhatla et al., (2003), Cell Transplant, 12:1-11. In another example, hepatocytes may be produced by culturing the TLR agonist-treated cells in serum-free medium in the presence of Activin A, followed by culturing the cells in fibroblast growth factor-4 and bone morphogenetic protein-2, e.g., Cai et al., (2007), Hepatology, 45(5): 1229-39. In an exemplary embodiment, the TLR agonist-treated cells are differentiated into hepatic cells or hepatic stem cells by culturing the TLR agonist-treated cells in the presence of Activin A from about 2 to about 6 days, e.g., about 2, about 3, about 4, about 5, or about 6 days, and then culturing the induced cells in the presence of hepatocyte growth factor (HGF) for from about 5 days to about 10 days, e.g., about 5, about 6, about 7, about 8, about 9, or about 10 days.

The TLR agonist-treated cells may also be differentiated into cardiac muscle cells. Inhibition of bone morphogenetic protein (BMP) signaling may result in the generation of cardiac muscle cells (or cardiomyocytes), see, e.g., Yuasa et al., (2005), Nat. Biotechnol., 23(5):607-11. Cardiomyocytes may be generated by culturing the TLR agonist-treated cells in the presence of leukemia inhibitory factor (LIF), or by subjecting them to other methods known in the art to generate cardiomyocytes from ES cells, e.g., Bader et al., (2000), Circ. Res., 86:787-794, Kehat et al., (2001), J. Clin. Invest., 108:407-414; Mummery et al., (2003), Circulation, 107:2733-2740.

Examples of methods to generate other cell-types from TLR agonist-treated cells include: (1) culturing induced cells in the presence of retinoic acid, leukemia inhibitory factor (LIF), thyroid hormone (T3), and insulin in order to generate adipocytes, e.g., Dani et al., (1997), J. Cell Sci., 110:1279-1285; (2) culturing TLR agonist-treated cells in the presence of BMP-2 or BMP4 to generate chondrocytes, e.g., Kramer et al., (2000), Mech. Dev., 92:193-205; (3) culturing the TLR agonist-treated cells under conditions to generate smooth muscle, e.g., Yamashita et al., (2000), Nature, 408:92-96; (4) culturing the TLR agonist-treated cells in the presence of beta-1 integrin to generate keratinocytes, e.g., Bagutti et al., (1996), Dev. Biol., 179:184-196; (5) culturing the TLR agonist-treated cells in the presence of Interleukin-3 (IL-3) and macrophage colony stimulating factor to generate macrophages, e.g., Lieschke and Dunn (1995), Exp. Hemat., 23:328-334; (6) culturing the TLR agonist-treated cells in the presence of IL-3 and stem cell factor to generate mast cells, e.g., Tsai et al., (2000), Proc. Natl. Acad. Sci. USA, 97:9186-9190; (7) culturing the TLR agonist-treated cells in the presence of dexamethasone and stromal cell layer, steel factor to generate melanocytes, e.g., Yamane et al., (1999), Dev. Dyn., 216:450-458; (8) co-culturing the TLR agonist-treated cells with fetal mouse osteoblasts in the presence of dexamethasone, retinoic acid, ascorbic acid, beta-glycerophosphate to generate osteoblasts, e.g., Buttery et al., (2001), Tissue Eng., 7:89-99; (9) culturing the TLR agonist-treated cells in the presence of osteogenic factors to generate osteoblasts, e.g., Sottile et al., (2003), Cloning Stem Cells, 5:149-155; (10) overexpressing insulin-like growth factor-2 in the TLR agonist-treated cells and culturing the cells in the presence of dimethyl sulfoxide to generate skeletal muscle cells, e.g., Prelle et al., (2000), Biochem. Biophys. Res. Commun., 277:631-638; (11) subjecting the TLR agonist-treated cells to conditions for generating white blood cells; or (12) culturing the TLR agonist-treated cells in the presence of BMP4 and one or more: SCF, FLT3, IL-3, IL-6, and GCSF to generate hematopoietic progenitor cells, e.g., Chadwick et al., (2003), Blood, 102:906-915.

In some cases, sub-populations of transdifferentiated somatic cells may be purified or isolated. In some cases, one or more monoclonal antibodies specific to the desired cell type are incubated with the cell population and those bound cells are isolated. In other cases, the desired subpopulation of cells expresses a reporter gene that is under the control of a cell type specific promoter.

The transdifferentiated cells may be used as a therapy to treat disease (e.g., a genetic defect). The therapy may be directed at treating the cause of the disease; or alternatively, the therapy may be to treat the effects of the disease or condition. The transdifferentiated cells may be transferred to, or close to, an injured site in a subject; or the cells can be introduced to the subject in a manner allowing the cells to migrate, or home, to the injured site. The transferred cells may advantageously replace the damaged or injured cells and allow improvement in the overall condition of the subject. In some instances, the transferred cells may stimulate tissue regeneration or repair.

The number of administrations of treatment to a subject may vary. Introducing the induced and/or differentiated cells into the subject may be a one-time event; but in certain situations, such treatment may elicit improvement for a limited period of time and require an on-going series of repeated treatments. In other situations, multiple administrations of the cells may be required before an effect is observed. The exact protocols depend upon the disease or condition, the stage of the disease and parameters of the individual subject being treated.

The cells may be introduced to the subject via any of the following routes: parenteral, intravenous, intraarterial, intramuscular, subcutaneous, transdermal, intratracheal, intraperitoneal, or into spinal fluid.

The transdifferentiated cells may be transferred to subjects suffering from a wide range of diseases or disorders. Subjects suffering from neurological diseases or disorders could especially benefit from cell therapies. In some approaches, the transdifferentiated cells are neural stem cells or neural cells transplanted to an injured site to treat a neurological condition, e.g., Alzheimer's disease, Parkinson's disease, multiple sclerosis, cerebral infarction, spinal cord injury, or other central nervous system disorder, see, e.g., Morizane et al., (2008), Cell Tissue Res., 331(1):323-326; Coutts and Keirstead (2008), Exp. Neurol., 209(2):368-377; Goswami and Rao (2007), Drugs, 10(10):713-719.

For the treatment of Parkinson's disease, the induced cells may be differentiated into dopamine-acting neurons and then transplanted into the striate body of a subject with Parkinson's disease. For the treatment of multiple sclerosis, neural stem cells may be differentiated into oligodendrocytes or progenitors of oligodendrocytes, which are then transferred to a subject suffering from MS.

Degenerative heart diseases such as ischemic cardiomyopathy, conduction disease, and congenital defects could benefit from stem cell therapies, see, e.g. Janssens et al., (2006), Lancet, 367:113-121.

Endothelial cells are useful in improving vascular structure and function, enhancing angiogenesis, and improving perfusion, e.g. in peripheral arterial disease.

Pancreatic islet cells (or primary cells of the islets of Langerhans) may be transplanted into a subject suffering from diabetes (e.g., diabetes mellitus, type 1), see e.g., Burns et al., (2006) Curr. Stem Cell Res. Ther., 2:255-266. In some embodiments, pancreatic beta cells derived from the methods of the invention cells may be transplanted into a subject suffering from diabetes (e.g., diabetes mellitus, type 1).

In other examples, hepatic cells or hepatic stem cells derived from induced cells are transplanted into a subject suffering from a liver disease, e.g., hepatitis, cirrhosis, or liver failure.

Hematopoietic cells or hematopoietic stem cells (HSCs) may be transplanted into a subject suffering from cancer of the blood, or other blood or immune disorder. Examples of cancers of the blood that are potentially treated by hematopoietic cells or HSCs include: acute lymphoblastic leukemia, acute myeloblastic leukemia, chronic myelogenous leukemia (CML), Hodgkin's disease, multiple myeloma, and non-Hodgkin's lymphoma. Often, a subject suffering from such disease must undergo radiation and/or chemotherapeutic treatment in order to kill rapidly dividing blood cells. Introducing HSCs derived from the methods of the invention to these subjects may help to repopulate depleted reservoirs of cells.

In some cases, hematopoietic cells or HSCs derived by transdifferentiation may also be used to directly fight cancer. For example, transplantation of allogeneic HSCs has shown promise in the treatment of kidney cancer, see, e.g., Childs et al., (2000), N. Engl. J. Med., 343:750-758. In some embodiments, allogeneic, or even autologous, HSCs derived from induced cells may be introduced into a subject in order to treat kidney or other cancers. Hematopoietic cells or HSCs derived from induced cells may also be introduced into a subject in order to generate or repair cells or tissue other than blood cells, e.g., muscle, blood vessels, or bone. Such treatments may be useful for a multitude of disorders.

It is to be understood that this invention is not limited to the particular methodology, protocols, cell lines, animal species or genera, constructs, and reagents described, as such may, of course, vary. It is also to be understood that the terminology used herein is for the purpose of describing particular embodiments only, and is not intended to limit the scope of the present invention, which will be limited only by the appended claims.

Unless defined otherwise, all technical and scientific terms used herein have the same meaning as commonly understood to one of ordinary skill in the art to which this invention belongs. Although any methods, devices and materials similar or equivalent to those described herein can be used in the practice or testing of the invention, the preferred methods, devices and materials are now described.

All publications mentioned herein are incorporated herein by reference for the purpose of describing and disclosing, for example, the reagents, cells, constructs, and methodologies that are described in the publications, and which might be used in connection with the presently described invention. The publications discussed above and throughout the text are provided solely for their disclosure prior to the filing date of the present application. Nothing herein is to be construed as an admission that the inventors are not entitled to antedate such disclosure by virtue of prior invention.

The following examples are put forth so as to provide those of ordinary skill in the art with a complete disclosure and description of how to make and use the subject invention, and are not intended to limit the scope of what is regarded as the invention. Efforts have been made to ensure accuracy with respect to the numbers used (e.g. amounts, temperature, concentrations, etc.) but some experimental errors and deviations should be allowed for. Unless otherwise indicated, parts are parts by weight, molecular weight is average molecular weight, temperature is in degrees centigrade; and pressure is at or near atmospheric.

### EXPERIMENTAL

### Example 1

### Activation of Innate Immunity in Non-Integrating Nuclear Reprogramming

Retroviral overexpression of the reprogramming factors (Oct4, Sox2, Klf4, c-Myc) generates induced pluripotential stem cells (iPSCs). However, the integration of foreign DNA could induce genomic dysregulation. One approach to overcoming this limitation is to express the factors as cell-permeant proteins (CPPs). To date this approach has proved difficult, and human somatic cells have not been reprogrammed using purified CPPs. We discovered a striking difference in the pattern of gene expression induced by viral versus protein-based delivery of the reprogramming factors, suggesting that a signaling pathway required for efficient nuclear reprogramming was activated by the retroviral, but not CPP approach. In both gain- and loss-of function studies, we find that activation of toll-like receptor 3 (TLR3) plays a role in the efficiency of nuclear reprogramming. Stimulation of TLR3 causes rapid changes in the expression of epigenetic modifiers, with chromatin remodeling and changes in gene expression that favor induction of pluripotency.

In seeking to develop effective reprogramming protocols for human cells via introduction of CPPs, we fortuitously observed an intriguing difference in the pattern of gene expression induced by viral as opposed to protein-based methods. The more rapid induction of gene expression that is observed with retroviral methodology is recapitulated by combining CPPs with retroviral particles encoding non-relevant genes, or more practically by combining CPPs with agonists of toll-like receptors (TLRs). We further observed that the induction of TLR3-mediated signaling promotes epigenetic remodeling that is required for efficient reprogramming. Recognition of the role of innate immunity in nuclear reprogramming, and its directed manipulation, provides key insights that may facilitate our understanding of both innate immunity and reprogramming and increase our understanding of the genetic and epigenetic pathways that function in induced pluripotency.

### Results

Different Patterns of Gene Expression Induced by Virus-Encoded Transcription Relative to Cell-Permeant Peptides

As previously described, our CPPs are fusion peptides, each with a reprogramming factor, a linker, and a cell transduction domain. As also described, these CPPs exhibit cognate DNA-binding activity, rapidly translocate across the plasma and nuclear membranes, uniformly transduce nearly all cells in the treated wells, and exert transcriptional control on known downstream target genes. Nevertheless, after multiple attempts with a variety of experimental protocols, we failed to generate iPSC lines from human fibroblasts using CPPs generated by our group or by commercial vendors.

In an effort to understand and overcome this failure, we examined the temporal sequence of target gene expression in response to a retroviral construct (pMX-Sox2) versus the corresponding CPP(CPP-SOX2). We focused on validated downstream targets such as Jarid2, Zic2, and bMyb for Sox2-activated genes, as well as, downstream genes known to function in nuclear reprogramming, i.e. Nanog, Sox2 and Oct4. Human fibroblasts were synchronized by serum starvation and then subjected to either a single infection with pMX-Sox2 or daily treatments of CPP-SOX2, and gene expression was assayed over 6 days. We used a daily dose of CPP-SOX2 (200 nM) that we had previously shown was capable of rescuing human iPSCs treated with shRNA against Sox2.

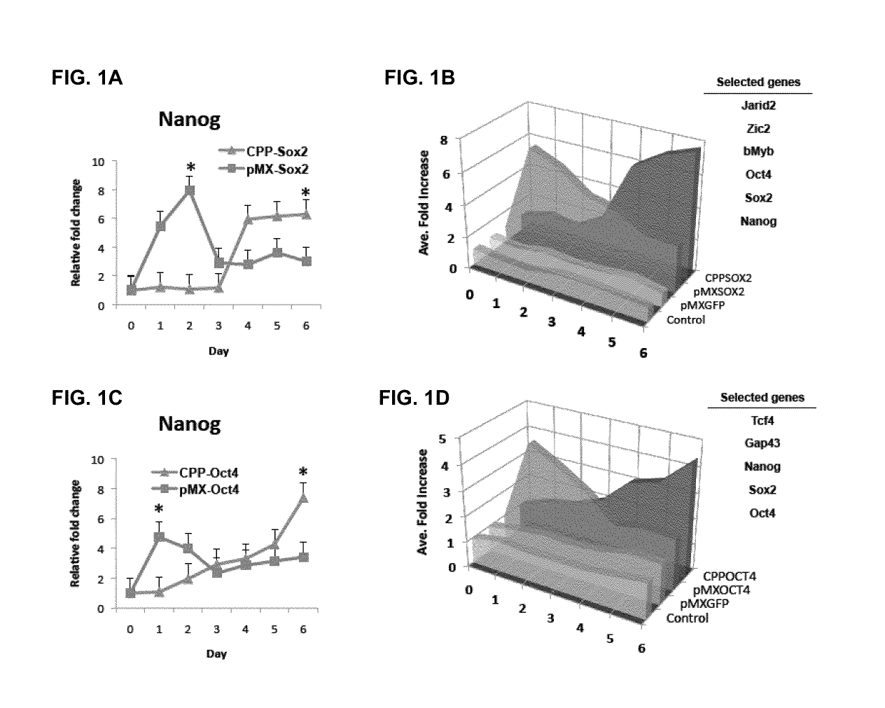

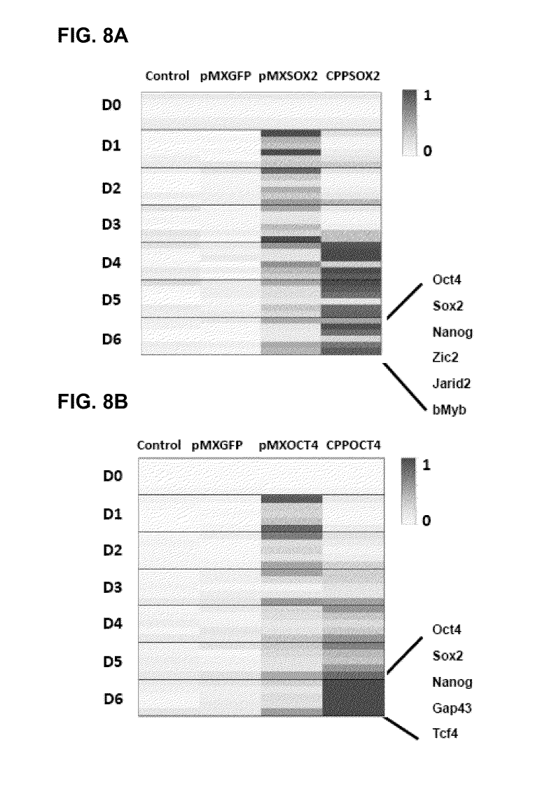

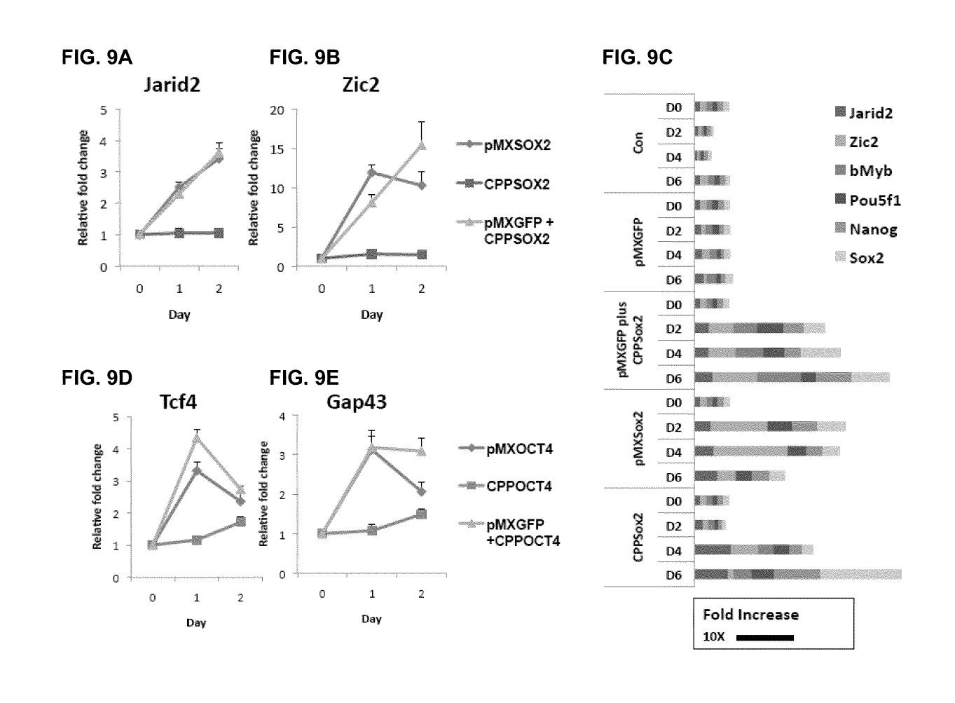

An intriguing difference in the pattern of gene expression was observed. As early as Day 1 of transfection with pMX-Sox2, human BJ fibroblasts manifested increased expression of the pluripotency (e.g. Nanog) and target genes (FIG. 1A and FIG. 8). By contrast, despite its rapid entry into the cytoplasm and nucleus of treated cells (within 2 h), CPP-SOX2 did not show a corresponding increase in target gene expression until several days later (FIG. 1A). Because the temporal pattern of expression of the selected genes (Jarid2, Zic2, bMyb, Oct4, Sox2 and Nanog) was remarkably similar for each treatment condition, their change in fold expression over time is shown as an average in FIG. 1B.

To exclude the possibility that the delay in target gene expression was a function of the design of a single CPP, we repeated these experiments comparing a CPP versus viral vector for Oct4 (pMX-Oct4), assessing their effect on downstream targets (Tcf4 and GAP43) and pluripotency genes. We observed a similar pattern of delayed gene expression in cells treated with CPPOCT4 compared to those transfected with pMX-Oct4 (FIGS. 1C-D). Again, because the temporal pattern of expression of the selected genes (Tcf4, GAP43, Oct4, Sox2 and Nanog) was remarkably similar for each treatment condition, their change in fold-expression over time is shown as a group average in FIG. 1D. These data reveal a profound difference between gene expression patterns in human fibroblasts exposed to reprogramming factors in a retroviral vector by comparison to those exposed to the reprogramming factors as purified CPPs.

Viral Particles Accelerate CPP-TF Induced Gene Expression

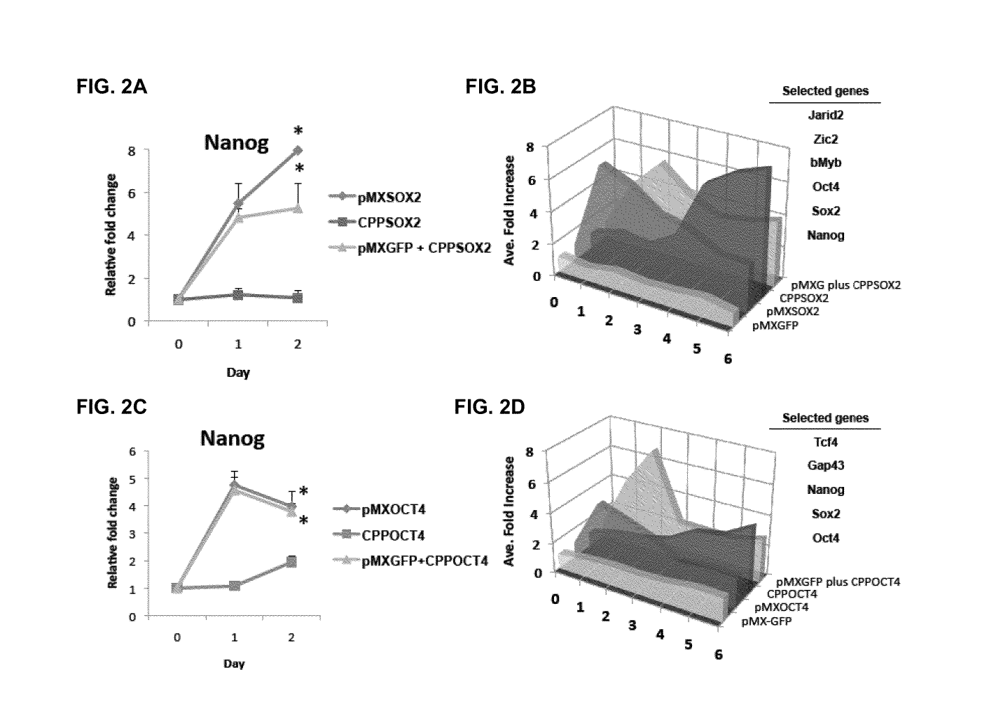

We hypothesized that an intrinsic feature of viral particles, independent of the genes encoded, might influence the reprogramming process. To test this hypothesis, we assessed the effect of the CPPs alone or in the presence of a retroviral particle encoding a gene not involved in reprogramming. The pMX-GFP control vector did not affect target gene expression (FIG. 8). However, when the pMX-GFP vector was combined with CPP-SOX2, the expression of the downstream genes was enhanced substantially, reproducing the pattern of gene expression induced by the retrovirus expression vector pMX-Sox2 (FIGS. 2A-B and FIGS. S2A-C). We repeated these studies with CPP-OCT4 (FIG. 2, and FIGS. S2D-E). When the pMX-GFP vector was combined with CPP-OCT4, again the temporal expression of the downstream genes was accelerated, mimicking the effect observed with the viral vector pMX-Oct4 (FIGS. 2C-D). These studies indicated that some intrinsic feature of the viral particle itself contributed to the effects of reprogramming factors on gene expression.

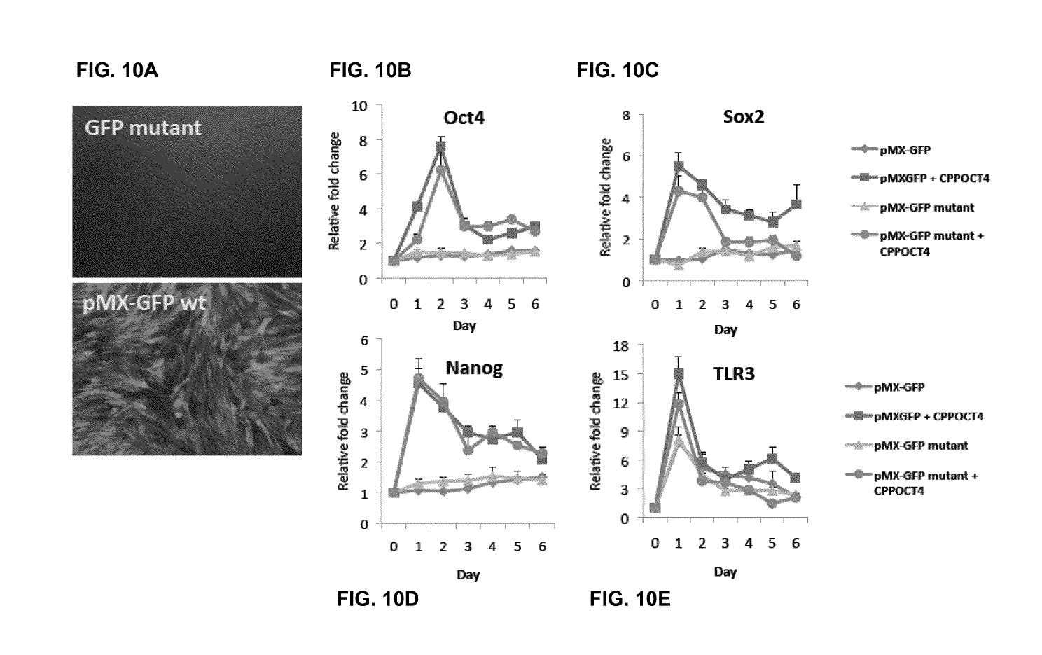

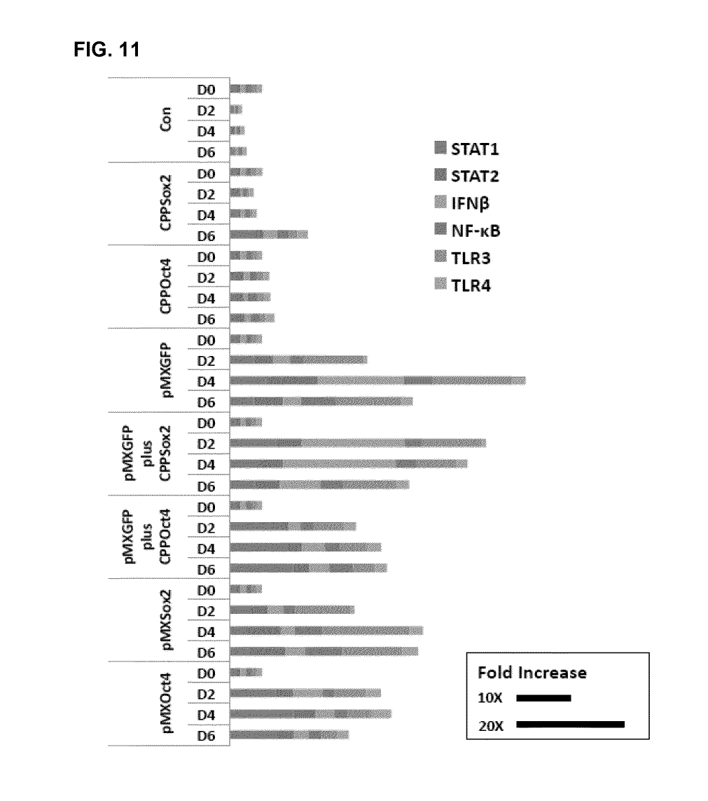

To gain more insight into the mechanisms by which a retroviral particle could accelerate nuclear reprogramming, we substituted a non-integrating mutant of pMX-GFP by introducing a frameshift mutation near the poi coding region of the retrovirus. This mutant can enter the cell, direct synthesis of intact virus particles that retain reverse transcriptase activity, but cannot integrate newly synthesized viral DNA into the host genome. Nevertheless, the pMX-GFP non-integrating mutant was fully capable of accelerating the gene expression induced by CPP-OCT4 (FIG. 10). Accordingly, integration of foreign DNA into the host genome is not required for the difference in gene expression between the CPPs and their corresponding viral vectors. Viral infection activates innate immunity, by virtue of interaction with Toll-like receptors (TLRs). The TLRs recognize pathogen-associated molecular patterns (PAMPs) associated with viral protein, lipopolysaccharides, DNA or RNA. We hypothesized that activation of innate immunity through TLRs may be involved in the difference in gene expression. Indeed, we observed that our retroviral vectors (but not the CPPs) activated inflammatory (innate immune response) genes, including the toll-like receptor 3 (TLR3), NF-κB, IFN-β, Stat1 and Stat2 (FIG. 11).

Knockdown of TLR3 Signaling Decreases Pluripotent Gene Expression Induced by Viral Vector Encoding Oct4.

The TLR-signaling pathway consists of two distinct pathways: a myeloid differentiation primary response gene (MyD) 88-dependent pathway, and a MyD88-independent pathway. The MyD88-dependent pathway is common to all TLRs, except TLR3. To distinguish which TLR signaling pathway might be involved in nuclear reprogramming with viral vectors, we used inhibitory peptides or shRNA knockdown directed against elements of the MyD88-dependent and -independent pathways. The TLR3 pathway is activated by viral dsRNA, and is independent of MyD88. The adaptor for TLR3 is TRIF (for TIR-domain-containing adapter-inducing interferon-β).

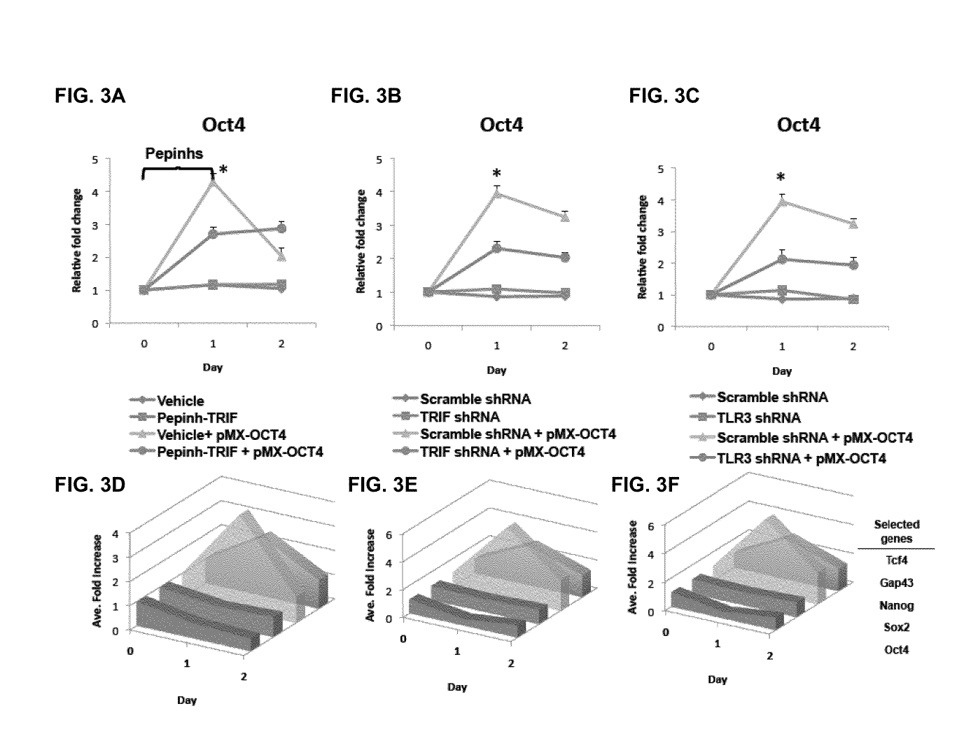

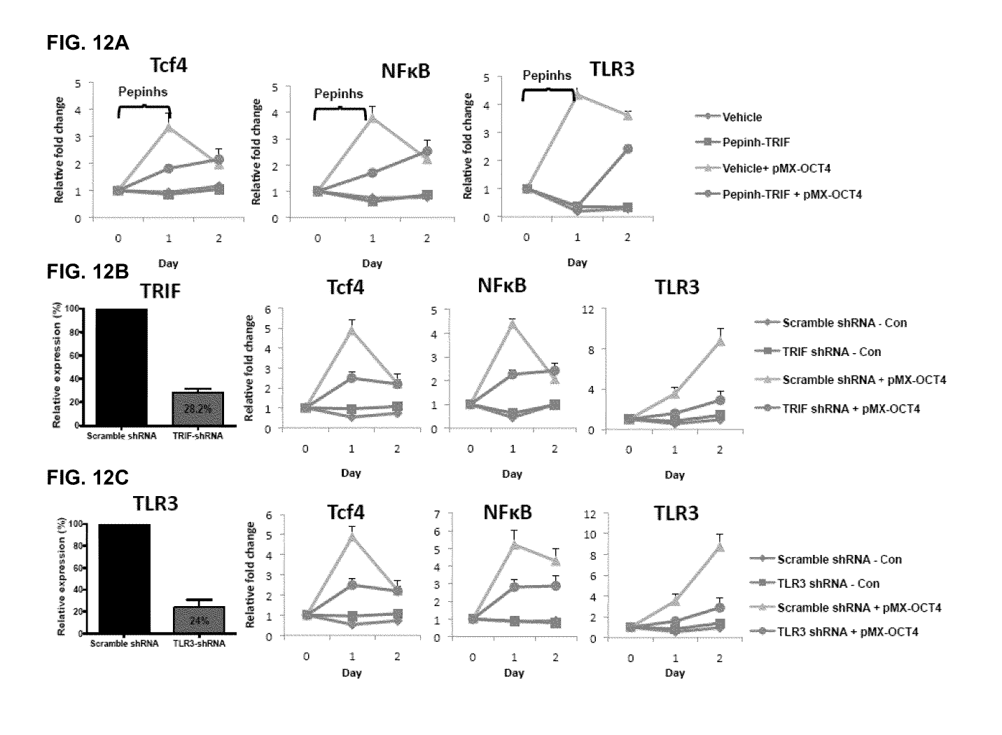

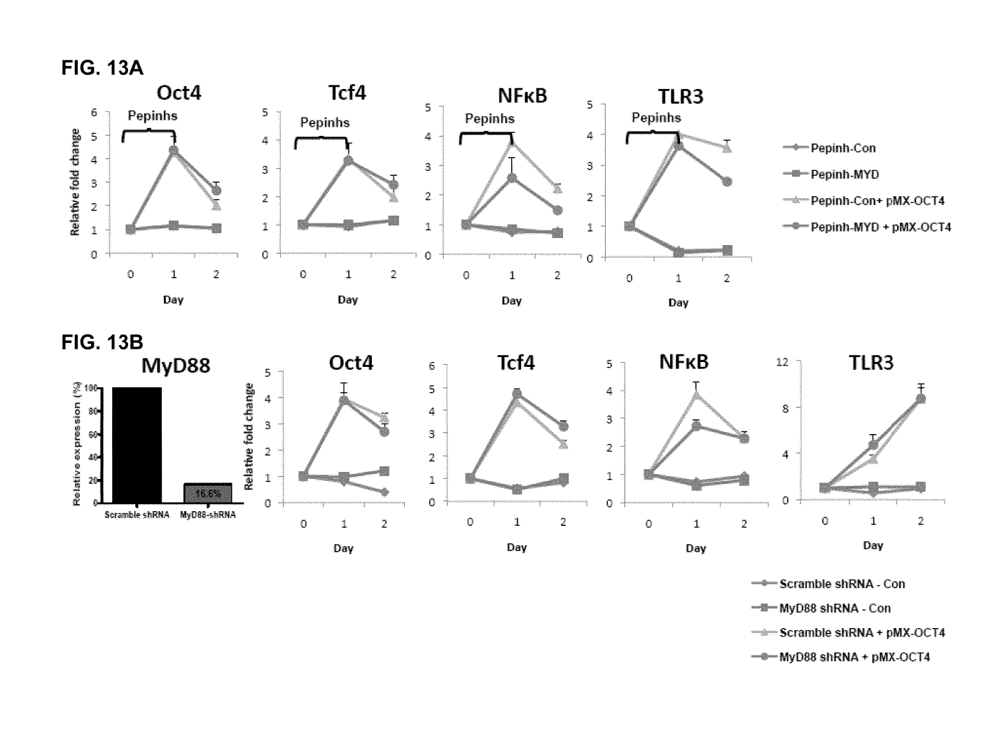

Accordingly, to explore the role of this pathway in the effect of the viral constructs on gene expression, we knocked down TLR3 or TRIF. In addition, we employed a cell permeable peptide inhibitor of TRIF. As expected, peptide inhibition of the TRIF adaptor molecule or knockdown by shRNA of TRIF or TLR3 significantly reduced the activation by pMX-Oct4 of immune response genes (FIG. 12). Notably, these knockdowns of TLR signaling also decreased the target and pluripotent gene expression induced by pMX-Oct4. The peptide inhibitor of TRIF (Pepin-TRIF) attenuated the effect of pMX-Oct 4 to induce Oct 4 expression (FIG. 3A), as well as expression of the other selected genes (FIG. 3D). Similarly, shRNA knockdown of TRIF (FIGS. 3B and 3E), as well as shRNA knockdown of TLR3 (FIGS. 3C and 3F) also attenuated the effect of pMX-Oct 4 to induce expression of the selected genes. By contrast, inhibition of MyD88 by an inhibitory peptide (FIG. 13A), or by a stable shRNA knockdown (FIG. 13B) had no effect on the target and pluripotent gene expression induced by pMX-Oct4. Together, these studies indicate that TLR3, but not the other TLR pathways, are required for full induction of target gene expression by the retrovirus expression vector.

TLR3 Signaling is Required for Efficient Generation of Human iPSCs

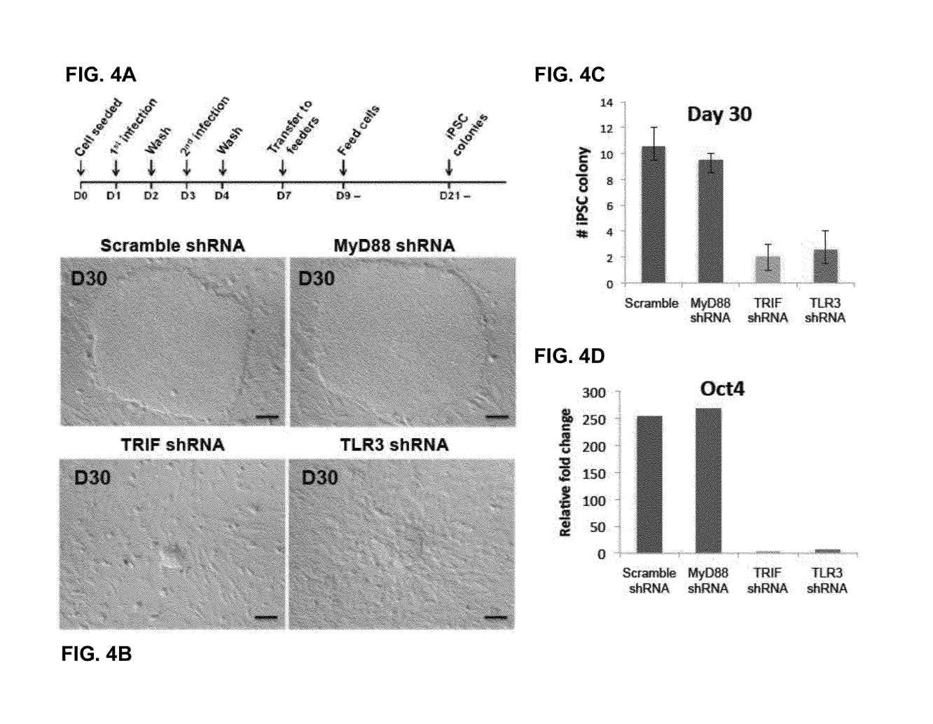

To determine if TLR3 signaling was necessary for efficient generation of human iPSCs, we exposed human BJ fibroblasts to retroviral vectors encoding OSKM into BJ fibroblast previously treated with scrambled shRNA or shRNA to knockdown (KD) the expression of TLR3, TRIF, or MyD88 (FIG. 4A). Six days following transduction, the cells were seeded on mitomycin C treated mouse embryonic fibroblasts (MEFs) and the following day, the medium was replaced with iPSC medium (containing 8 ng/ml basic FGF). Around day 25 we observed small colonies in the Scrambled- and MyD88-KD cells; by contrast, no colonies were observed in the TRIF- or TLR3-KD cells. By day 30 distinct colonies with typical iPSC colony morphology were noted in dishes containing the scramble- and MyD88-KD cells (FIG. 4B). At this time, the TRIF- and TLR3-KD cells manifested only small granulated colonies. It took another 9 days for the TRIF and TLR3-KD cells to yield morphologically distinct iPSC colonies. We manually counted each distinct colony with typical morphological features, as well as those smaller granulated colonies, appearing from day 30 to day 39 (in two independent experiments by an observer blinded to the treatment group). As seen in FIG. 4C, at early time points the TRIF- and TLR3-KD cells generated significantly fewer colonies by comparison to scramble- and MyD88-KD cells. Furthermore, we compared the gene expression values between these colonies at day 30. The expression of Oct4 (FIG. 4D), Sox2 and Nanog were upregulated more than 10-fold when compared to the TRIF- and TLR3-KD iPSCs. These findings provided the first evidence that TLR3 activation is necessary for efficient induction of pluripotential genes and generation of human iPSC colonies using the approach first described by Yamanaka.

TLR3 Agonist Accelerates CPP-Induced Target Gene Expression

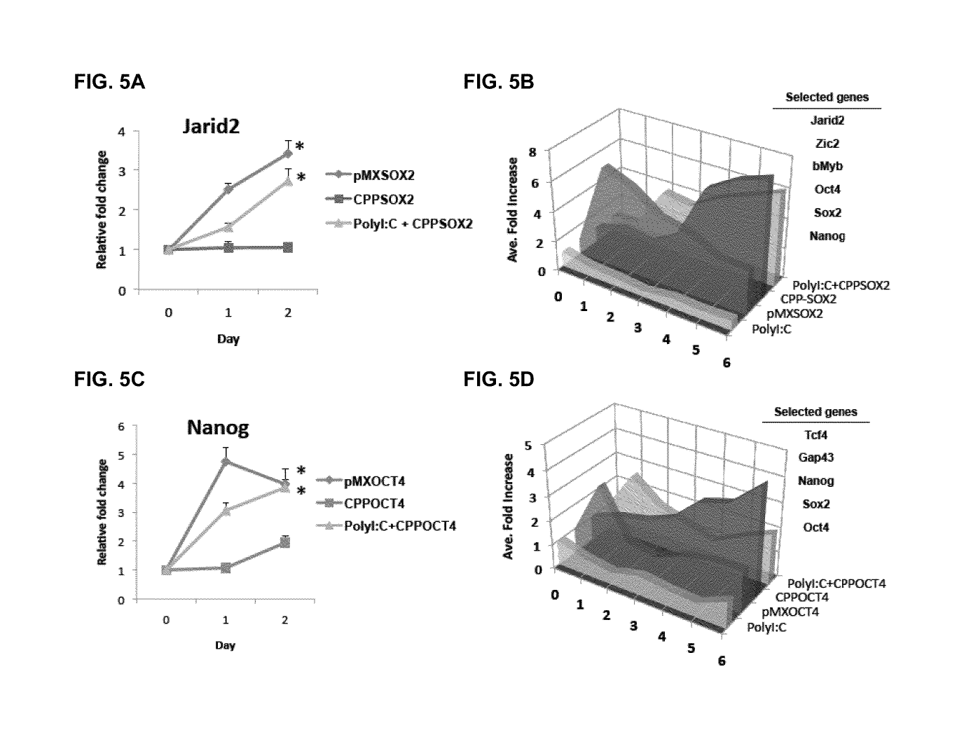

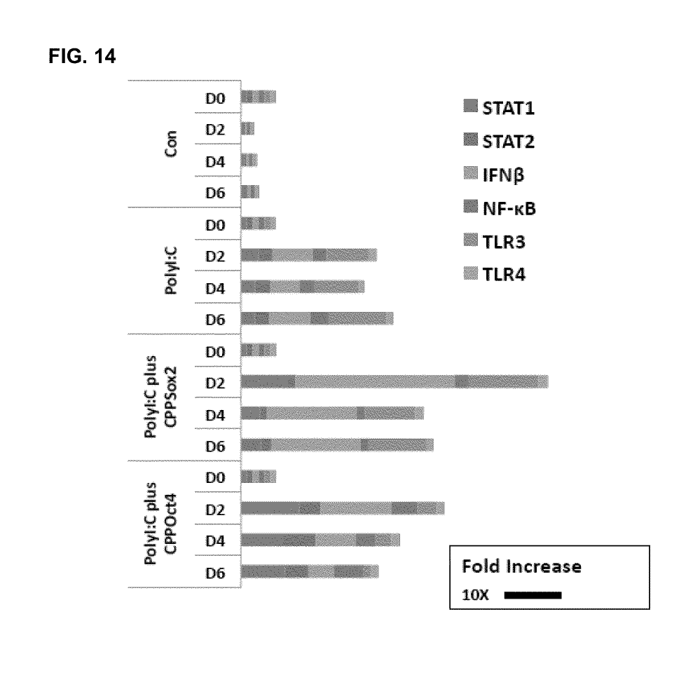

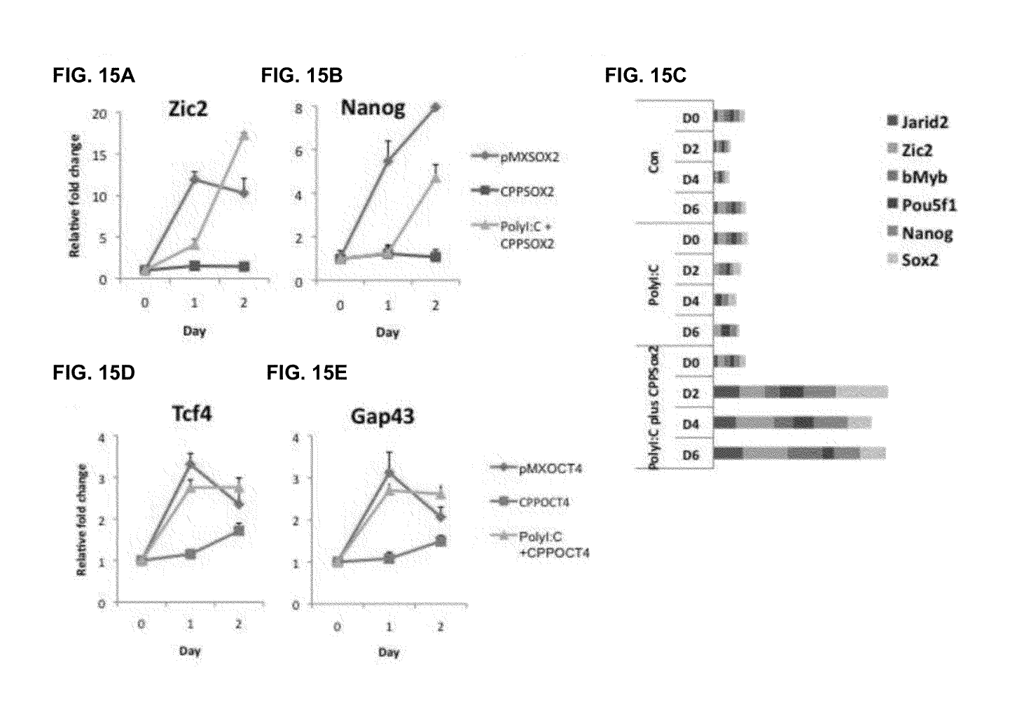

If TLR3 activation plays a role in the efficiency of viral-based reprogramming, then the addition of a TLR3 agonist would be predicted to enhance CPP-induced reprogramming. Polyinosinicpolycytidylic acid (Poly I:C) is a synthetic analog of dsRNA that is recognized specifically by TLR3 and which induces the expression of genes involved in innate immunity (FIG. 14). Accordingly, we assessed the effect of the CPPs alone or in the presence of poly I:C. The expression of target genes was unaffected by poly I:C alone. However, when poly I:C (300 ng/ml) was combined with CPP-SOX2, the expression of the downstream genes was accelerated, reproducing the time course of gene expression induced by pMX-Sox2 (FIGS. 5A-B; and FIGS. 15A-C). We repeated these studies with CPP-OCT4. When poly I:C was combined with CPP-OCT4, again the temporal expression of the downstream genes was accelerated, mimicking the effect observed with the viral vector pMX-Oct4 (FIGS. 5C-D; and FIGS. 15D-E). These studies provided further support for the hypothesis that TLR3 signaling is required for efficient induction of the target genes of the reprogramming factors.

TLR3 Activation Enhances Efficiency of a Doxycycline-Inducible System for Generating iPSCs

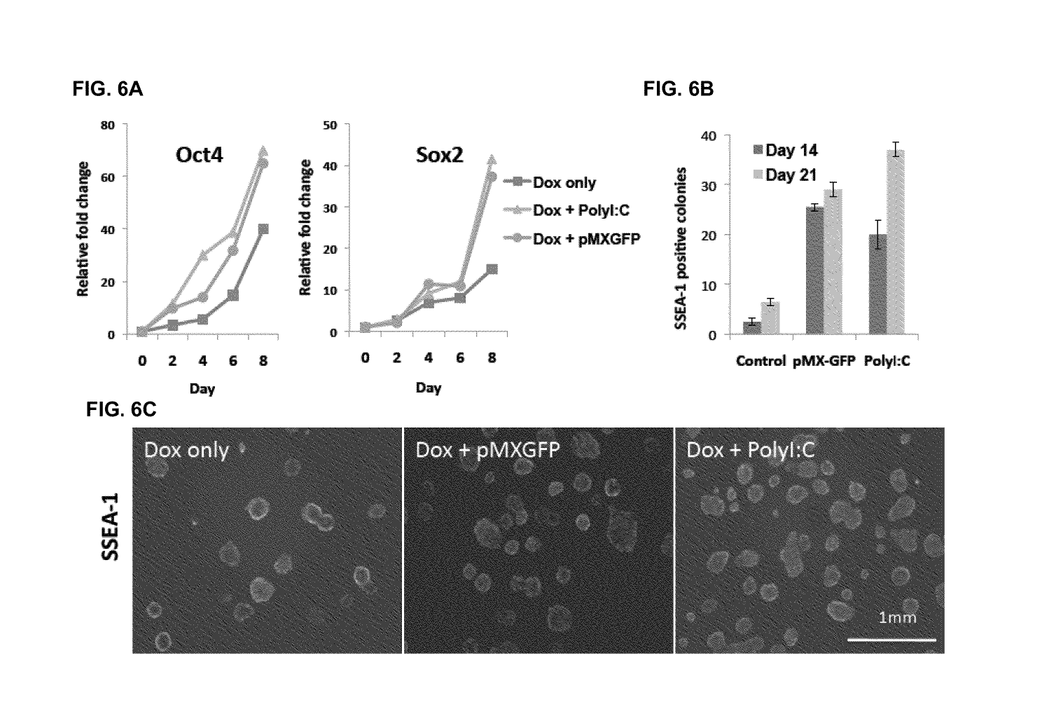

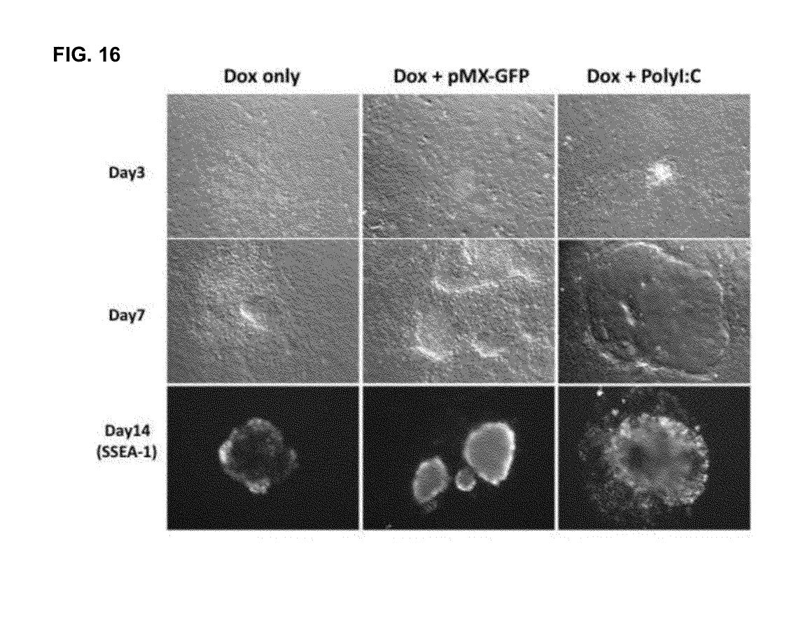

To further test the hypothesis that TLR3 activation was required for efficient reprogramming, we isolated MEFs from murine embryos expressing a doxycycline (Dox)-inducible polycistronic transgene construct encoding the four reprogramming factors. To generate iPSCs, 10⁵ MEFs/per well in 6-well plates were treated with Dox. In some wells, poly I:C was also added for the initial 6 days of the reprogramming process. In other wells, cells were infected with pMX-GFP on the first day of Dox treatment. Poly I:C or pMX-GFP each accelerated the expression of Oct4 and Sox2 (FIG. 6A). Poly I:C as well as pMX-GFP accelerated changes in the morphology of the MEFs with small, compact rounded cells aggregating in the wells at 3 days (FIG. 16). Similarly, infection with pMX-GFP seemed to accelerate colony formation, as a number of small colonies were observed by day 7 in the viral particle infected group (FIG. 16). By day 14, typical mES-like colonies appeared, many of which had activated SSEA-1. At this time point, the number of typical SSEA-1⁺ colonies were increased by 7-8 fold in wells exposed to viral particles or poly I:C (FIG. 6B). Colony number increased further by day 21-28 (FIGS. 6B and 6C). These studies demonstrated that TLR3 activation enhances nuclear reprogramming to pluripotency.

TLR3 Activation Enhances CPP-Induced Generation of Human iPSCs

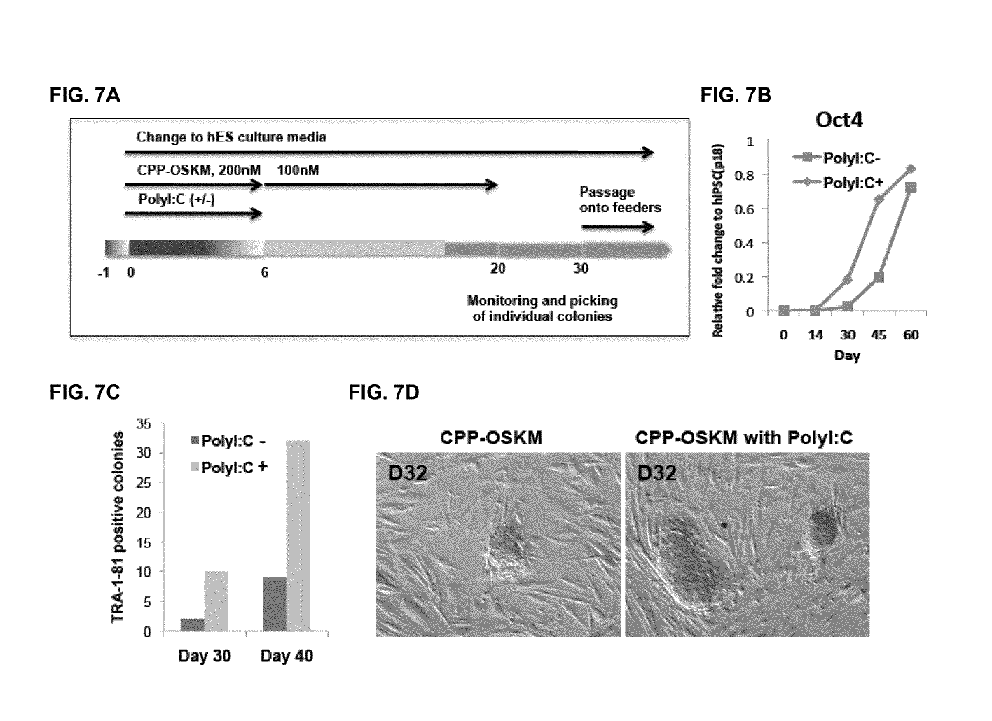

It is known that persistent expression (about 2 weeks) of the reprogramming factors is required using viral vectors to generate mouse iPSCs. However, we failed to generate human iPSCs even after continuous exposure to the CPPs for 6-30 days. We hypothesized that activation of the TLR3 pathway might facilitate epigenetic alterations required for full transcriptional effect of the CPPs. We also attempted to mimic the biphasic effect of the reprogramming factors introduced as viral vectors by reducing the dose of CPPs after 6 days. Accordingly, we exposed human fibroblasts to the four CPP-transcription factors (Oct4-R11, Sox2-R11, Klf4-R11 and cMyc-R11) (at a dose of 200 nM for days 1-6, and a dose of 100 nM for days 7-21), in the presence or absence of poly I:C (300 ng/ml) for days 1-6 (FIG. 7A). The cultures were transferred to feeder cells (inactivated MEFs) at day 26. In the presence of poly I:C, Oct4 expression was accelerated (FIG. 7B). Furthermore, poly I:C accelerated iPSC generation (FIGS. 7C-D). Small colonies were observed by day 21 and ES like colonies appeared by day 30, many of which expressed TRA-1-81 as indicated by live cell staining. By contrast, in human fibroblasts not exposed to poly I:C, colonies were not observed until day 30 days. By day 40, colony number was increased by more than 4-fold in cells exposed to poly I:C (FIG. 7C). Application of our insights regarding the role of TLR3 signaling in nuclear reprogramming permitted us to successfully reprogram human fibroblasts to pluripotency using CPPs.

TLR3 Activation Causes Epigenetic Changes that Favor Reprogramming

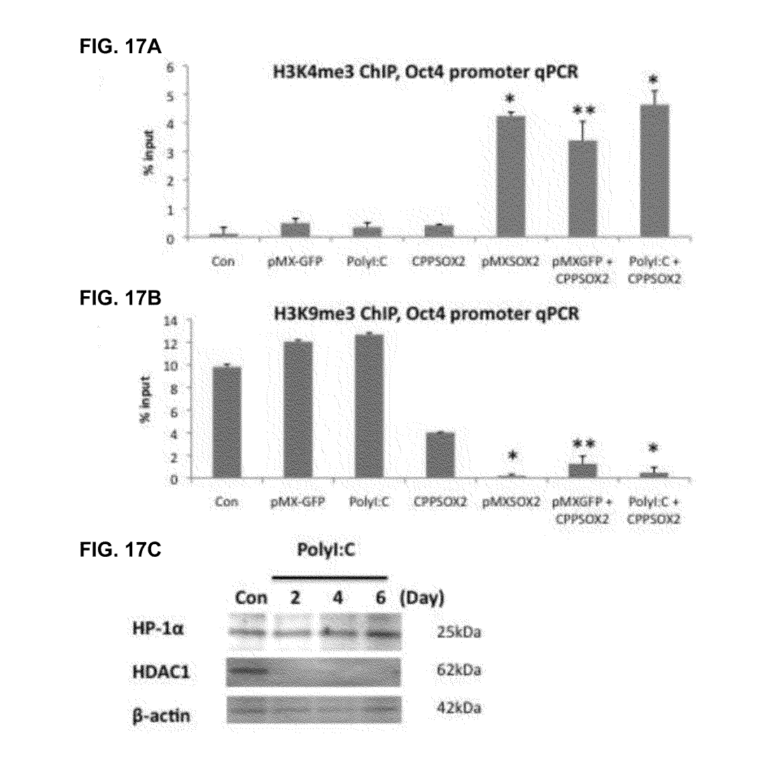

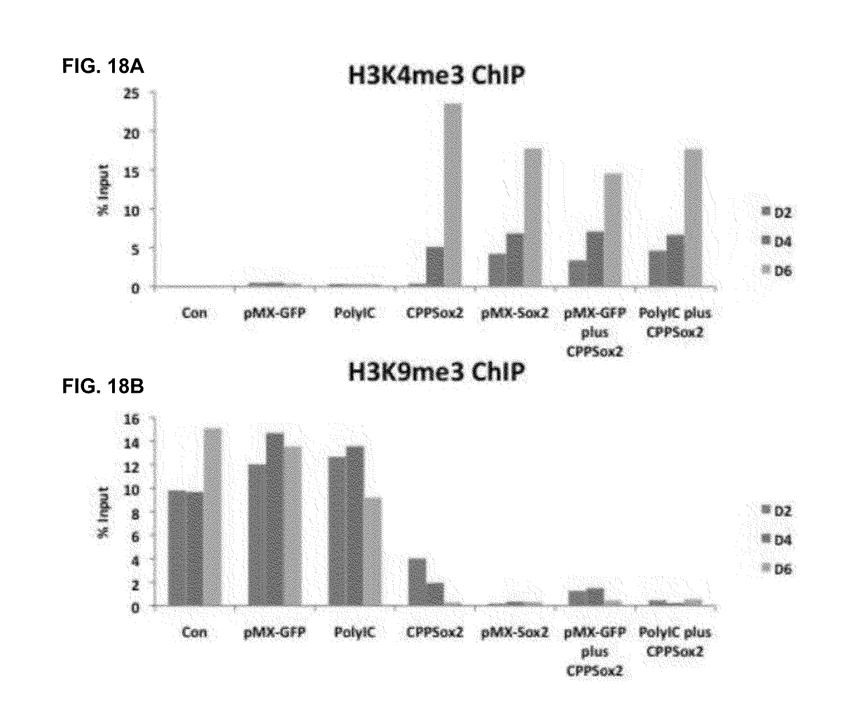

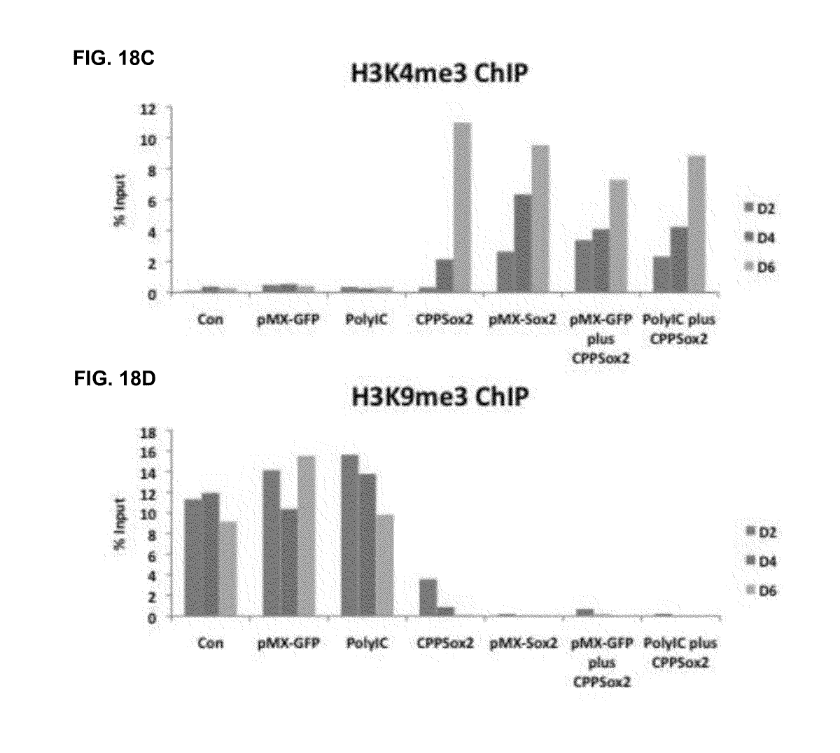

We hypothesized that TLR3 activation might enhance early transcriptional activation by inducing an open chromatin state, permitting the reprogramming factors to induce an ESC-specific gene expression pattern. Accordingly, we performed ChIP assays to detect trimethylation of histone H3 at lysine 4 (H3K4me3). This epigenetic modification marks transcriptionally active genes. Human fibroblasts were treated with pMXSox2, or with CPPSox2 in the presence of poly I:C or pMXGFP. By day 2 of treatment, pMXSox2 but not CPPSox2 alone, induced H3K4 trimethylation at the Oct4 promoter (FIG. 8A). However, in the presence of viral vector or poly I:C, CPPSox2 induced changes in H3K4 trimethylation on day 2 (FIG. 17). Although CPPSox2 alone could induce H3K4 trimethylation (FIG. 18A), it was only after a time lag that reflected its delayed effects on target gene expression. Similarly, poly I:C, or the retroviral vector encoding GFP, accelerated H3K4 trimethylation at the Sox2 promoter in CPP-treated fibroblasts (FIG. 18C). In a similar fashion, we assessed histone H3 at lysine 9 (H3K9me3) in the Oct4 and Sox2 promoters. This epigenetic modification marks transcriptionally silenced genes. By day 2 of treatment, pMXSox2 but not CPPSox2 alone, fully reversed H3K9 trimethylation at the Oct4 promoter (FIG. 17). Poly I:C or pMXGFP enhanced the H3K9 trimethylation induced by CPPSox2 on day 2 (FIG. 17). Although CPPSox2 alone could fully reverse H3K9 trimethylation at the Oct4 promoter (FIG. 18B), it was only after a time lag that reflected its delayed effects on target gene expression. Similarly, poly I:C, or the retroviral vector encoding GFP, accelerated the loss of H3K9 trimethylation at the Sox2 promoter in CPP-treated fibroblasts (FIG. 18D). These studies provided an epigenetic mechanism to explain the effect of TLR3 activation to enhance nuclear reprogramming.

TLR3 Activation Regulates Epigenetic Machinery: Role of NE-κB

Histone acetylation status influences the folding and functional state of the chromatin and modulates the accessibility of DNA to the transcriptional machinery for gene expression. Histone de-acetylation is generally associated with a closed chromatin state, and inhibitors of histone de-acetylase (HDAC) such as valproic acid are employed to enhance nuclear reprogramming. Therefore it is notable that poly I:C downregulated the expression of a suite of HDAC genes in CPP treated human fibroblasts. The downregulation of HDAC1 expression by poly I:C was confirmed by Western analysis (FIG. 17). Similar downregulation of the HDAC family by poly I:C was noted in the dox-inducible MEFs described above and in FIG. 6.

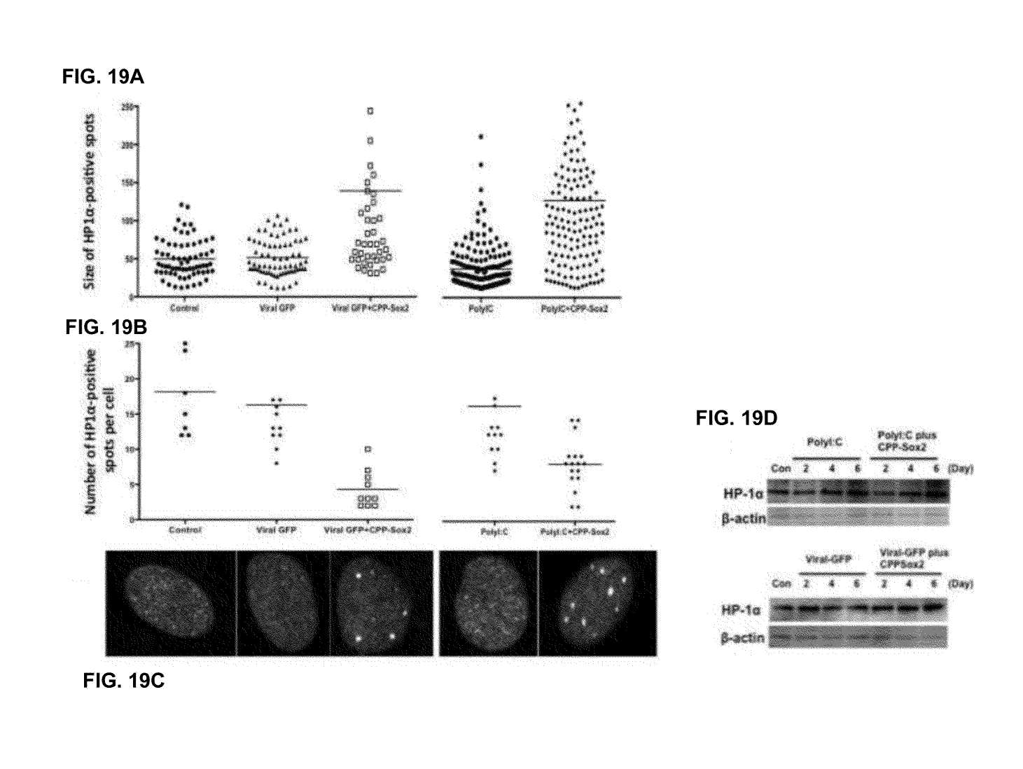

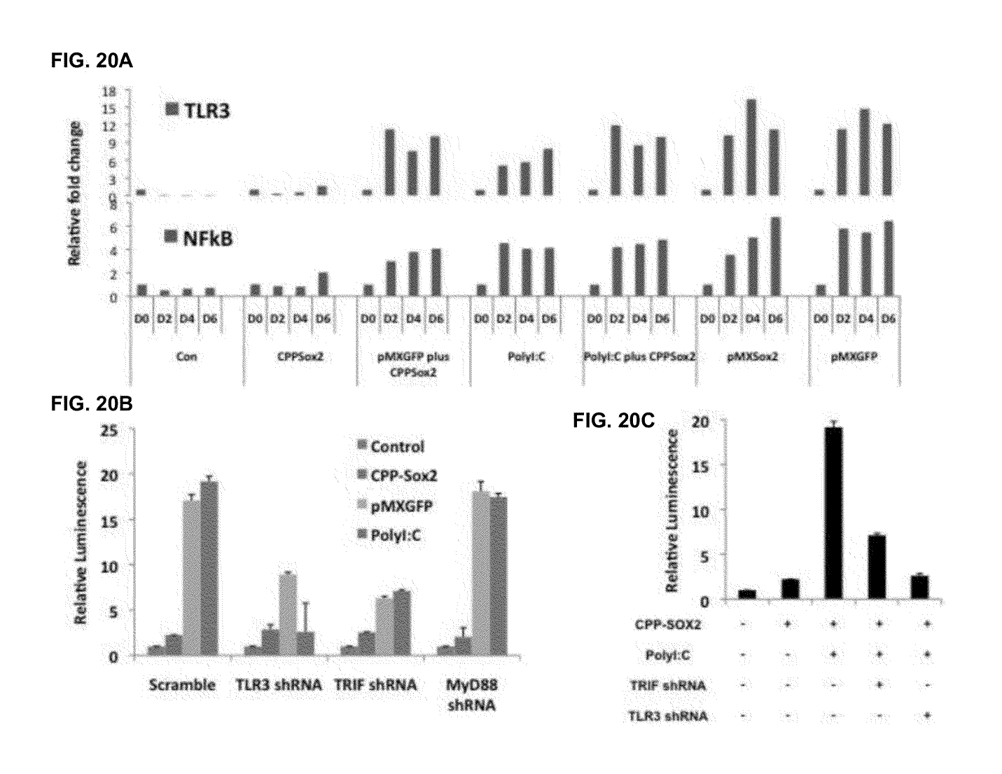

Poly I:C significantly affected the expression of other epigenetic modifiers. In addition, the changes in methylation status of the Oct4 and Sox2 promoters that were accelerated by TLR3 activation were also associated with an accelerated redistribution of heterochromatin protein 1 (HP1; FIGS. 19A and 19B). HP1 that is bound to methylated H3K9 recruits the methylase Suv39h, leading to further methylation of H3K9, so as to consolidate a repressed state. The redistribution of HP1 induced by poly I:C is consistent with genome-wide epigenetic alterations induced by TLR3 activation. Histone acetylation favors an open chromatin state, maintained by proteins containing histone acetyltransferase (HAT) domains, such as p300 and CBP. NF-κB is a transcriptional effector of TLR3 activation, and interacts with CBP/p300 to positively regulate gene expression. We used a luciferase reporter assay system to document that poly I:C, but not the CPPs alone, induced NF-κB activation (FIG. 20). This effect was mediated by TLR3, as shRNA knockdown of TLR3 or its adaptor protein TRIF1 reduced the effect of poly I:C on NF-κB activation (FIG. 20). Similarly, the retroviral constructs, as well as poly I:C, induced a sustained increase in the expression of NF-κB and TLR3, whereas CPP-SOX2 did not (FIG. 20). These studies suggest that the effect of TLR3 activation to induce changes in gene expression of the epigenetic machinery might be mediated in part by NF-κB.

In seeking to induce pluripotency while employing cell permeant peptides (CPPs), we serendipitously discovered a role for innate immunity signaling in effective nuclear reprogramming. Our salient observations are: 1.) A consistent difference in the temporal characteristics of gene expression is observed between cells exposed to the reprogramming factors in the form of retroviral vectors versus CPPs; 2.) TLR3 knockdown inhibits the activation of downstream target genes when using retroviral vectors to overexpress the reprogramming factors, and reduces the efficiency and yield of human iPSC generation; 3.) TLR3 activation accelerates the expression of downstream target genes using CPPs; enhances the efficiency and yield of miPSC generation in a dox-inducible system; and enhances the efficiency and yield of human iPSC generation when using the reprogramming factors in the form of CPPs, and 4.) TLR3 activation induces epigenetic alterations, including changes in methylation status of the Oct4 and Sox2 promoters, as well as changes in the expression of epigenetic effectors, that promote an open chromatin configuration. This report is the first to posit a direct link between nuclear reprogramming efficiency and inflammatory pathways in the induction of pluripotency.

An Unappreciated Role for TLR3 Activity in Reprogramming.

TLR3 recognizes double-stranded RNA (dsRNA) generated by retroviruses. The importance of TLR signaling for effective nuclear reprogramming has not been appreciated. We show that the efficiency and yield of human iPSC generation, using retroviral vectors, is reduced by knockdown of the pathway with peptide inhibitors or shRNA knockdown of TLR3 or its adaptor protein TRIF. We confirmed the importance of TLR signaling in a virus free system using murine embryonic fibroblasts that were genetically engineered to express a doxycycline-inducible cassette encoding the reprogramming factors. In this system, the coadministration of the TLR3 agonist poly I:C increased the efficiency and yield of murine iPSCs (FIG. 6). Notably, the same effect was observed with co-administration of the retrovirus encoding GFP, which retrovirus would be expected to activate TLR3 without otherwise affecting the nuclear reprogramming process. Our work indicates that the retroviral vectors used for inducing pluripotency are more than vehicles for delivering the reprogramming factors, and actively contribute to the reprogramming process.

TLR3 Activation Enhances Reprogramming.

The knowledge that the activation of innate immune response affects nuclear reprogramming permitted us to enhance the efficiency and yield of human iPSCs using reprogramming factors in the form of CPPs. Heretofore, human somatic cells have not been reprogrammed to pluripotency using purified CPPs. Human iPSCs have been generated using extracts derived from HEK cells overexpressing the Yamanaka factors. However, it is likely that these cell extracts contain factors (e.g. viral DNA) that may trigger inflammatory pathways. That said, we learned that it was possible to achieve nuclear reprogramming with CPPs alone. This success was only achieved after we modified our experimental protocol so as to mimic the biphasic gene expression pattern observed with the retroviral administration of the reprogramming factors (i.e. we reduced the dose of administered CPP, starting on day 6).

TLR3 and Epigenetic Modification.

The effect of TLR3 activation to enhance the yield and efficiency of human iPSC generation appears to be due in part to its regulation of the expression or distribution of epigenetic modifiers. We used cDNA profiling to examine the effect of TLR3 activation. We observed repression of the histone deacetylase family, with significant reductions in expression of HDAC 1, 2, 5, and 7. We also observed downregulation of the methyltransferases SMYD1, PRMT 2, 6 and 8; the histone-lysine-N-methyltransferase ASH11; and the serine/threonine-protein kinase Nek6. Associated with the changes in expression of epigenetic modifiers, we observed histone modifications consistent with an open chromatin configuration on the promoter regions of Oct4 and Sox2 (FIGS. 17, 18). Notably however, the increase in H3K4 trimethylation and the decrease in H3K9 trimethylation of these promoters were not observed with TLR3 activation alone. Only in the presence of the CPPs did poly I:C induced the changes in these methylation marks. This observation shows that, although TLR 3 activation causes widespread changes in the expression of epigenetic modifiers that might promote the open chromatin configuration of pluripotency genes, the reprogramming proteins are likely necessary to direct the epigenetic modifiers to the appropriate promoter sequences. This notion is also supported by the confocal images of HP1a distribution (FIGS. S19A-C).

Heterochromatin protein-1 (HP1) is associated with the closed conformation of chromatin. Although we did not see changes in the expression levels of HP1 expression (FIG. 19D), we observed marked changes in its distribution when CPP-SOX2 was co-administered with poly I:C or with the retroviral vector encoding GFP. However, in the absence of the CPP, there was no observable redistribution of HP1 induced by poly I:C or the retroviral vector alone. Any of five classes of pathogen recognition receptors (PRRs) have been shown to signal in ways that are comparable to TLR3, and might be expected to accelerate nuclear reprogramming. The fact that the current work implicated TRIF signaling likely aligns with the fact that the retrovirus RNA provides adequate TLR3 signaling to trigger NF-κB, IRF3 and IFN. While TLR3 mimicked signaling from the retrovirus vector, other TLR agonists as well as agonists for NOD-like receptors, RIG-1-like receptors, cytosolic DNA sensors and C-type lectin receptors, drive a similar inflammatory response converging on NF-κB, IRF-3 and IFNβ.

To conclude, our observations highlight a previously unrecognized role for innate immunity activation in nuclear reprogramming. The vectors used to induce pluripotency are more than mere vehicles for the reprogramming factors. Their stimulation of the TLR3 receptor induces epigenetic activation that accelerates the action of the reprogramming factors on their downstream target genes. Recognition of the role of innate immunity signaling in nuclear reprogramming may lead to insights that enhance the efficiency and quality of reprogramming and advance the therapeutic application of iPSCs.

### Experimental Procedures

Cells BJ human fibroblast cells derived from foreskin (Stemgent) were cultured in DMEM with 10% FBS and 1% penicillin/streptomycin (pen-strep) antibiotics in a humidified 5% CO₂ incubator at 37° C. For MEF isolation, chimeric embryos were isolated at E13.5 from single-gene transgenic R26_{rtTA}; Col1a1_{2lox-4F2A} mice expressing the IoxP-flanked, dox-inducible polycistronic 4F2A cassette (Oct4, Sox2, Klf4, c-Myc) from the Col1a1 locus obtained from Jackson Laboratory. After removal of the head and internal organs, the remaining tissues were physically dissociated and incubated in trypsin at 37° C. for 20 min, after which cells were re-suspended in MEF media containing puromycin (2 μg/ml) and expanded for two passages before freezing.

Viral preparation and infection HEK293FT cells were plated at 6×10₆ cells per T225 flask and incubated overnight. Cells were transfected with 10 μg of VSV-G (envelope protein), 15 μg of pUMVC (packaging plasmid) and 10 μg of gene of interest (Sox2 or Oct4) with Lipofectamine. 48 hours after transfection, the supernatant of transfectant was collected and filtered through a 0.45 μm filter. Following spinning at 17,100 rpm for 2 hr 20 min, the viral pellet was resuspended to make 100× stock solutions. Human fibroblasts were seeded at 5×10₄ cells per well of a 6-well dish a day before transduction. The medium was replaced with virus-containing supernatant supplemented with 8 μg/ml polybrene, and incubated for 24 hr.

Treatments At 60-70% confluency, BJ fibroblast cells were serum-starved using 1% serum to induce G1 cell cycle arrest. The synchronized BJ fibroblasts were then subjected to either a single infection with retroviral constructs or daily treatments with 200 nM CPPs (CPP-SOX2 or CPP-OCT4). Poly I:C (300 ng/ml) was added to the cells simultaneously with the CPPs. For experiments involving peptide inhibitors, cells were pretreated for 6 hrs at 40 uM with either MyD88 inhibitory peptide (Pepinh-MyD) or TRIF inhibitory peptide (Pepinh-TRIF) followed by CPP treatments.

Gene Expression and Microarray Analyses RNA was isolated with the RNeasy kit. First-strand cDNA was primed with oligo(dT) primers and qPCR was performed with primer sets from Applied Biosystems. RNA probes were prepared and hybridized to Illumina HumanHT-12 v4 Expression BeadChip microarrays.

Short Hairpin RNA Design Short hairpin RNA was obtained from Invivogen. Target sequences: MyD88 shRNA, AACTGGAACAGACAAACTATC; TRIF shRNA, AAGACCAGACGCCACTCCAAC and TLR3 shRNA, GCTTGGCTTCCACAACTAGAA

Chromatin Immunoprecipitation and ChIP-qPCR qChIP was performed as previously described (Lim et al., 2009; Peng et al., 2009). For qChIP and qRT-PCR, error estimates are standard deviations. Recovery of genomic DNA as the percentage input was calculated as the ratio of copy numbers in the immunoprecipitate to the input control. Primers of Oct4 and Sox2 promoters were purchased from Cell Signaling.

Generation of iPSCs Retroviral-iPSCs: As described previously (Takahashi et al., 2007; Takahashi and Yamanaka, 2006), human fibroblasts previously treated with MyD88, TRIF, TLR3 or Scramble shRNA were transduced with pMX-Oct4, Sox2, Klf4, and cMyc retroviruses and were cultured in iPSC medium on mitomycin-treated MEFs. Colonies were counted over time, and were harvested for RNA isolation qPCR analysis for pluripotent gene expression.

Protein-iPSCs: Recombinant Oct4, Sox2, Klf4, and cMyc human proteins (CPPs) contained an eleven-arginine membrane penetration domain at the C terminus were obtained from Stemgent. Human fibroblasts were treated with CPPs encoding the reprogramming factors (CPP-Oct4, CPP-Sox2, CPP-Klf4 and CPP-Myc) daily for 6 days with 200 nM CPPs, followed by daily treatments of 100 nM CPPs from day 7 to day 20. Poly I:C (300 ng/ml) or vehicle was added to the cell simultaneously only up to day 6. The cells were passed onto MEF feeders at day 30. After 20 days of CPP treatments, wells were closely scanned for colonies.

Doxycycline-induced iPSCs: As previously described (Wernig et al., 2008), MEFs from chimeric embryos at E13.5 were isolated. 4×10₄ secondary MEFs (passage #4) were plated per well in six-well plates and treated with doxycycline (2 μg/mL)±poly I:C (300 ng/ml). The generation of iPSC colonies was monitored daily and scored at days 14 and 21.

NF-κB Luciferase assay BJ fibroblasts (3×10₅) were seeded in a 6-well plate and subjected to either pMX-GFP infection, CPP-SOX2 treatment with or without poly IC (300 ng/ml). Cells were transfected with pNF-κB-Luc and pFC-MEKK as a positive control plasmid using Lipofectamine 2000. Twentyfour hours post-transfection, cells were collected for measuring the luciferase activity by the Bright-Glo™ Luciferase Assay System using a luminometer.

Immunostaining of Live Cells For the detection of SSEA-1 or TRA-1-81 in live cells, the primary Ab (anti-mouse SSEA-1, antihuman TRA-1-81, Stemgent) was diluted to a final concentration of 2.5 to 5 μg/ml in fresh cell culture medium and incubated with cells for 30 minutes at 37° C. and 5% CO2. After gentle washing the cells were examined under a fluorescent microscope.

Western Blotting Proteins were extracted from BJ fibroblasts by solubilizing the cells in RIPA buffer containing 1× protease inhibitor cocktail. 25 μg of total protein was loaded and resolved on SDSpolyacrylamide gels, transferred to PVDF membranes and probed with the following primary antibodies: anti-HP1α and anti-HDAC (Cell Signaling), and β-actin (Sigma, A5441). Immunoblots were developed with enhanced chemiluminescence reagents (Amersham).

### Example 2

### Direct Reprogramming of Fibroblasts to Functional Endothelial Cells (ECs)

As disclosed above, it has been demonstrated that ECs can be derived from ESC or iPSCs, and that these pluripotent stem cell-derived ECs can enhance limb perfusion and angiogenesis in murine models of PAD. However, other sources of differentiated cells, such as autologous ECs, are also highly desirable. For clinical applications in particular, it is desirable to develop strategies involving minimal use of genetic manipulation, i.e. non-integrating factors. Innate immunity (for example, via toll-like receptors) pathway plays an important role in nuclear reprogramming and importantly, when activated can cause rapid and global changes in the expression of epigenetic modifiers to enhance chromatin remodeling.

With the recognition of the role of innate immunity in nuclear reprogramming, and its directed manipulation to favor an open chromatin state, we hypothesized that activation of TLR3, together with external microenvironmental cues that drive EC specification, can induce the transdifferentiation of fibroblasts into ECs.

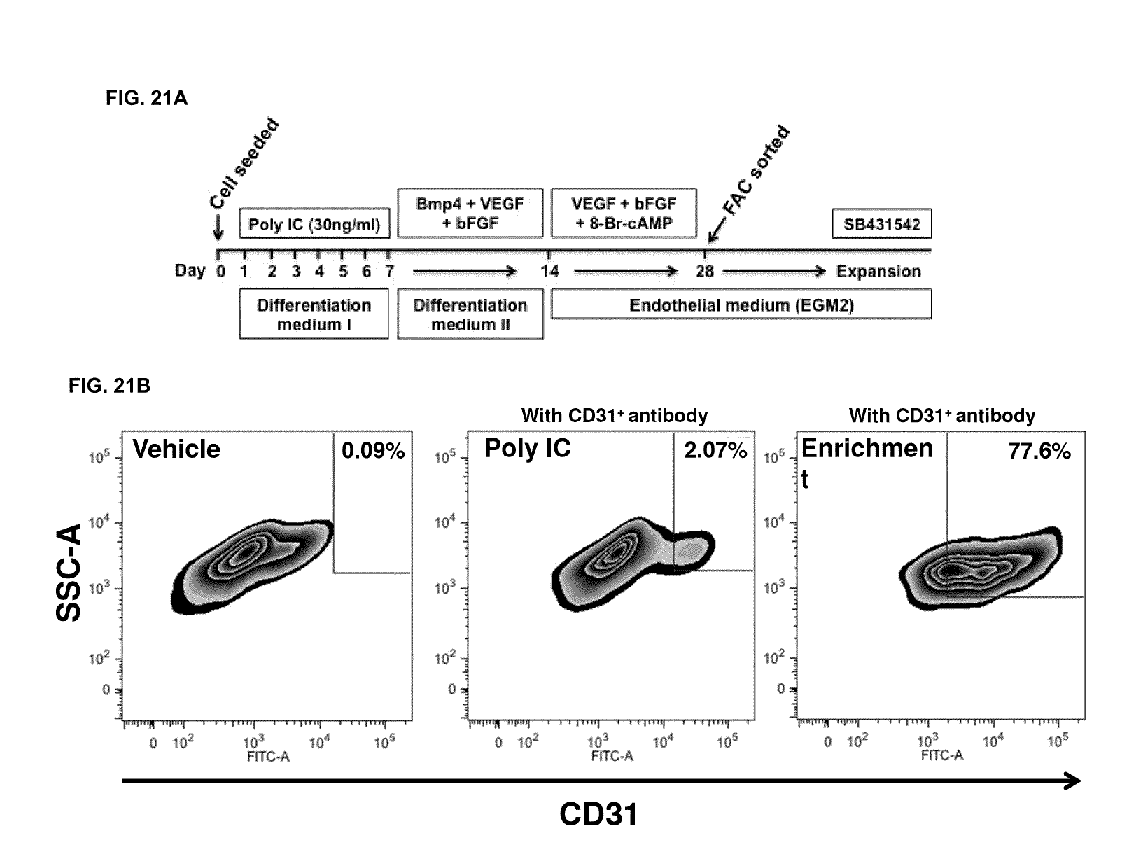

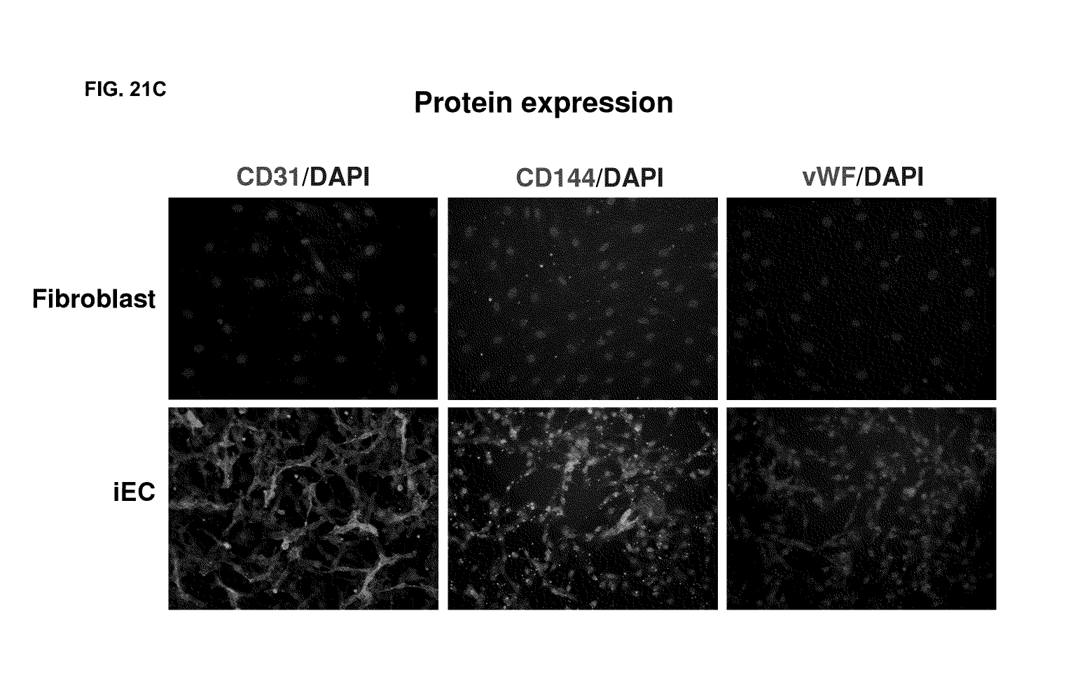

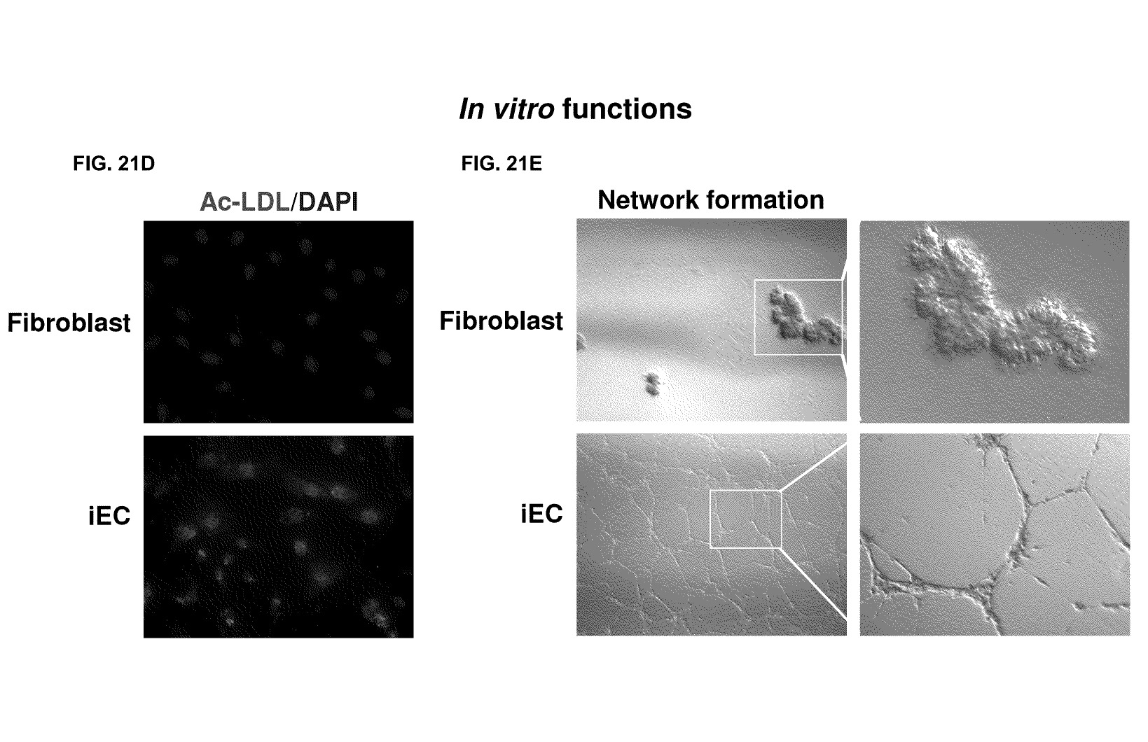

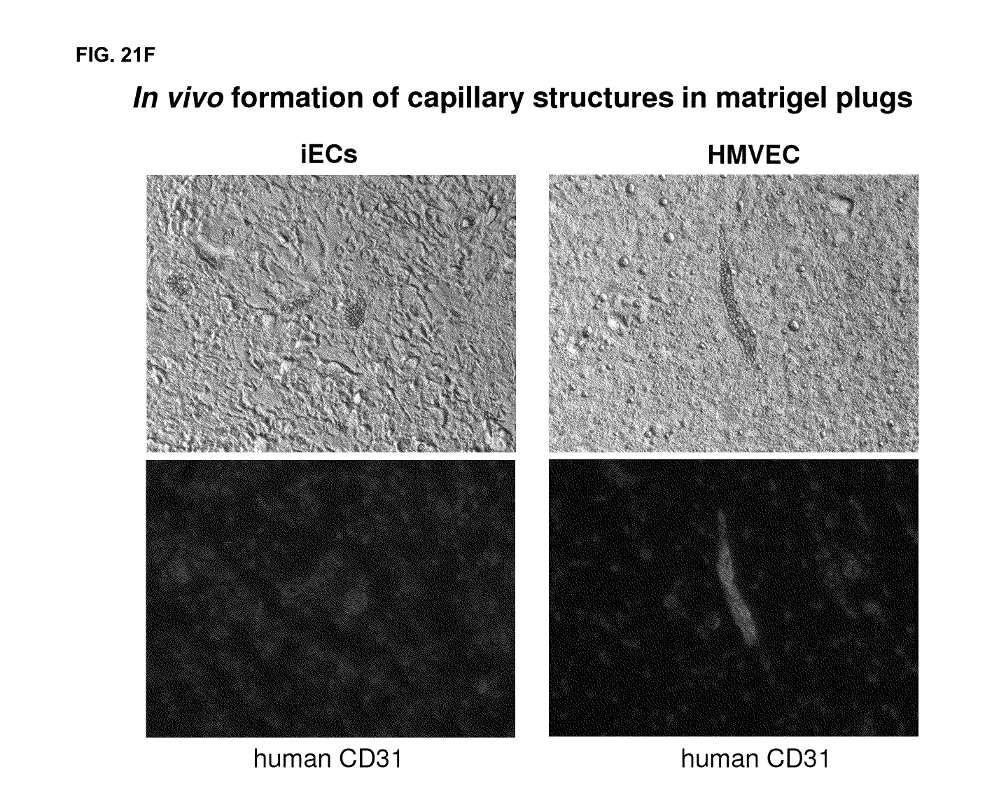

To determine if human fibroblasts could be converted to ECs via activation of innate immunity, human foreskin fibroblasts (BJ) were treated with Poly I:C (30 ng/ml) and cultured in a mixture of fibroblast medium and defined growth medium containing knockout serum (FIG. 1A). After culture for 7 days, the medium was changed to differentiation induction medium, supplemented with bFGF (20 ng/ml), VEGF (50 ng/ml) and BMP4 (20 ng/ml), which are known to promote induction of an endothelial lineage. To further increase the efficiency of endothelial transdifferentiation, we added 8-Br-cAMP (an agonist of cyclic AMP-dependent protein kinase to our protocol, as it enhances endothelial specification. After 28 days of differentiation, the cells were dissociated and purified for EC-specific marker VE-cadherin or CD31 by Fluorescence-activated cell sorting (FACS). Approximately 2% of cells expressed CD31, relative to the vehicle control (FIG. 21B). To further enhance the expansion of induced endothelial cells (iECs), we added SB431542, a specific TGF receptor inhibitor that promotes ESC-derived endothelial cell growth and sheet formation. After expansion, the iECs were sorted to show 77% purity for VE-cadherin or CD31 (FIG. 21B).

After expansion, the iECs formed a typical “cobblestone” monolayer, and continued to express endothelial markers, including CD31, VE-cadherin, KDR, Von Willebrand factor (vWF) and eNOS. Similarly, immunofluorescence staining revealed that these iECs were positive for EC markers such as CD31, VE-cadherin and vWF (FIG. 21C). Furthermore, these iECs were able to incorporate acetylated LDL and form networks of tubular structures on matrigel (FIG. 21D-E). In addition, these iECs showed the capacity to form capillaries when injected subcutaneously after placing them in matrigel and adding growth factor VEGF (FIG. 21F).

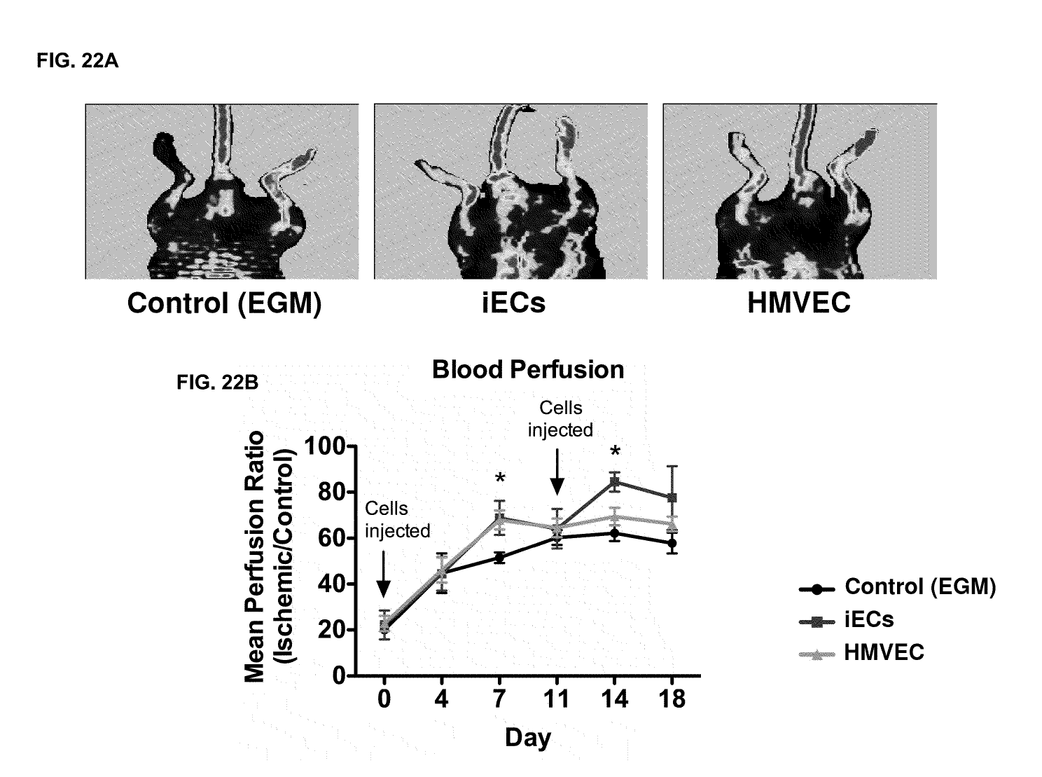

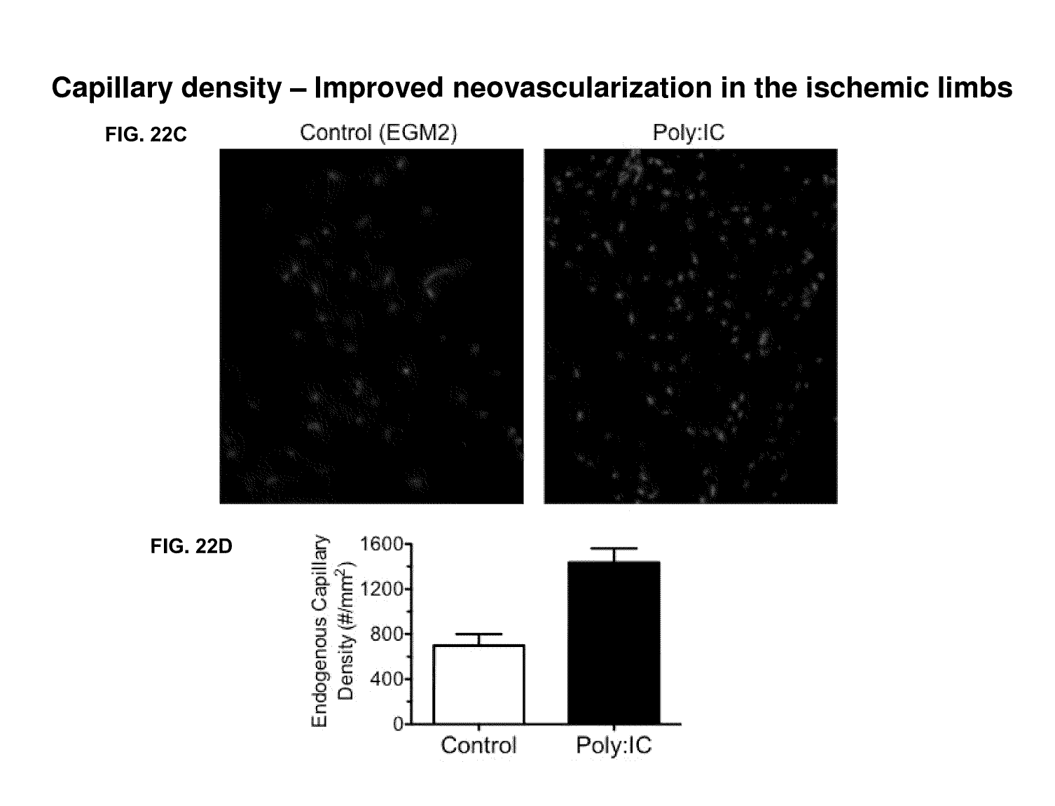

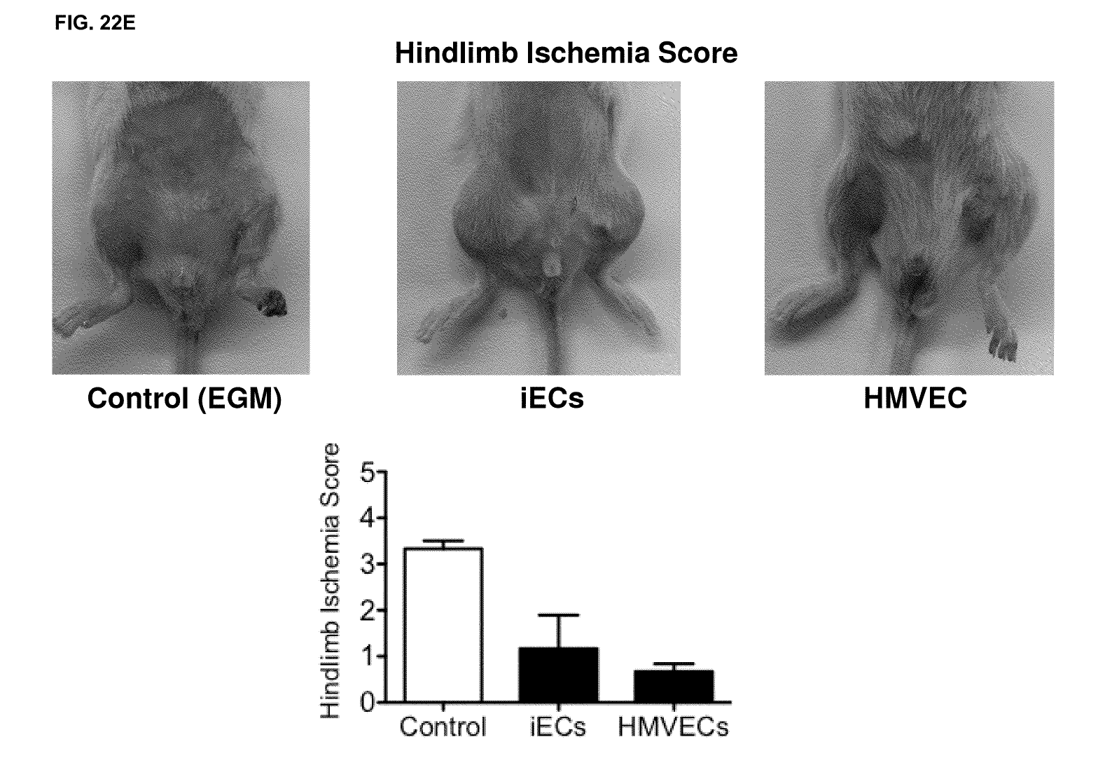

Therapeutic potential of iECs in a model of peripheral arterial disease: (iECs improve blood perfusion in a mouse model of peripheral artery disease). To functionally characterize iECs and to determine their capacity for vascular regeneration, we employed the hindlimb ischemic model in mice in which ischemia was induced by ligating the femoral artery of NOD SCID mice. The mice were then assigned to receive intramuscular injection (to the gastrocnemius muscle) either iECs, human ECs or vehicle. To analyze subcutaneous hindlimb perfusion, laser Doppler perfusion imaging analysis was performed to determine the effects of transplantation of iECs on the ischemia hindlimb. The hindlimb perfusion ratio (ischemic/control hindlimb) was significantly improved in the iEC-treated mice compared to the vehicle-treated mice (FIG. 22A-B). To confirm the laser Doppler data, the sections of ischemic hindlimbs at day 18 were stained with mouse CD31 antibody to assess capillary density by immunofluorescence staining. Mouse CD31 positive capillary density was significantly greater in the iEC group compared to the control group (FIGS. 22C&D). Furthermore, the hindlimb ischemia was assessed by blinded observers to obtain a hindlimb ischemia score, which revealed a significant improvement of blood perfusion and regeneration in iEC-treated mice (FIG. 22E).

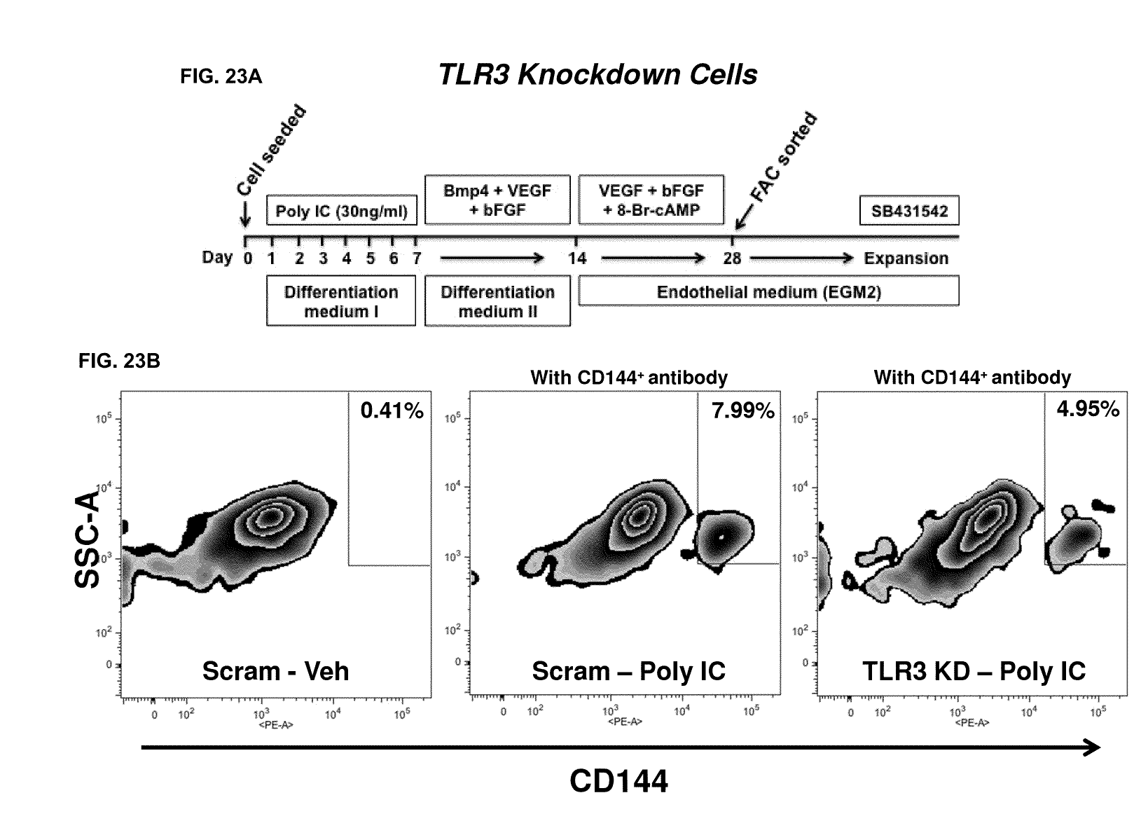

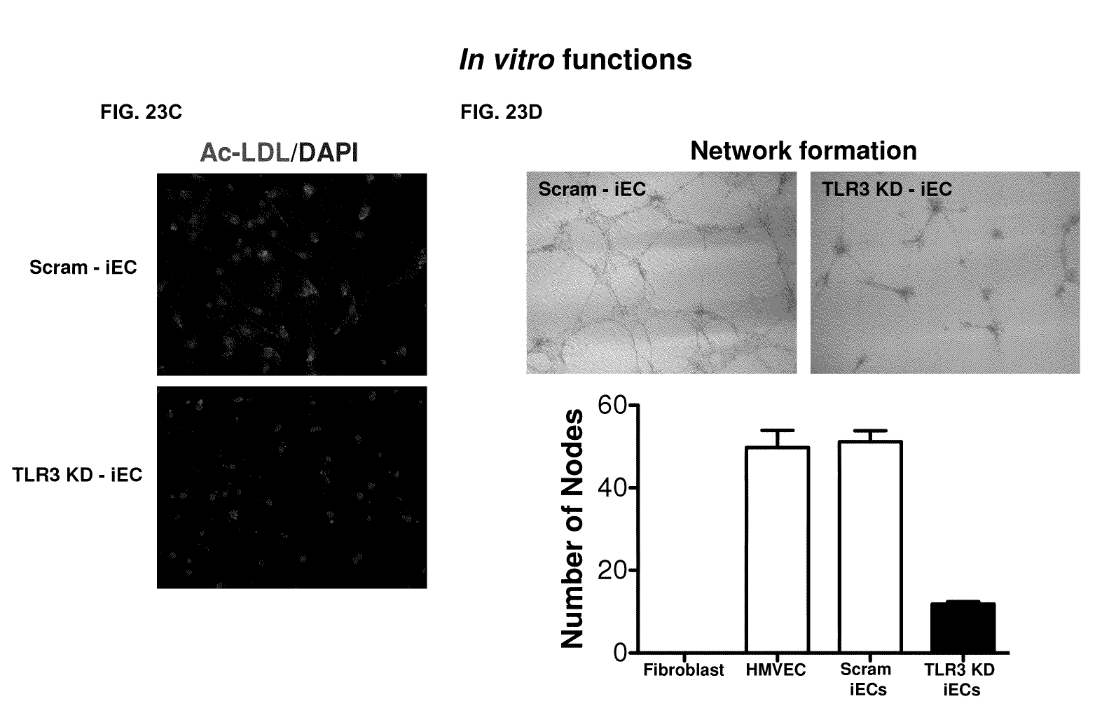

Innate immunity (TLR3 signaling) enables efficient transdifferentiation of fibroblasts to ECs: To determine whether TLR3 signaling was necessary for efficient transdifferentiation of human fibroblasts, we assessed direct differentiation in BJ fibroblasts previously treated with scrambled shRNA or shRNA to knockdown (KD) the expression of TLR3. Following treatment with Poly I:C (30 ng/ml) and chemically defined differentiation medium, cells were cultured in EC specific medium supplemented with growth factors (bFGF, VEGF and BMP4). Following 28 days of differentiation, cells were dissociated and FAC sorted for VE-cadherin (FIG. 23A). As seen in FIG. 23B, scramble treated cells generated significantly more iECs compared to TLR3-KD cells when treated with Poly I:C. Even though the iECs generated from the TLR3KD cells showed only a modest reduction in gene and protein expression for EC markers when compared to scramble, they showed a significant decrease in their capacity to uptake acetylated LDL (FIG. 23C) and form networks of tubular structures on matrigel (FIG. 24D). To further elucidate the elements of TLR3 signaling involved in transdifferentiation, we inhibited the action of downstream effector NFκB. NF-κB is a transcriptional effector of TLR3 activation and interacts with CBP/p300 to positively regulate gene expression. We found that Poly I:C significantly enhanced the transdifferentiation of fibroblasts to iECs. This effect of Poly I:C to enhance transdifferentiation (FIG. 23E), was markedly reduced by the addition of p65 decoy suggesting that TLR3-induced activation of NFkB is involved in direct reprogramming.

Huang, N. F. et al. Arteriosclerosis, Thrombosis, and Vascular Biology 30, 984-991. Rufaihah, A. J. et al. Arteriosclerosis, Thrombosis, and Vascular Biology 31, e72-e79, (2011). Margariti, A. et al. Proceedings of the National Academy of Sciences 109, 13793-13798, (2012). Yamamizu, K., Kawasaki, K., Katayama, S., Watabe, T. & Yamashita, J. K. Blood 114, 3707-3716, (2009). Watabe, T. et al. The Journal of Cell Biology 163, 1303-1311, (2003).

What is claimed is:

1. A method of nuclear reprogramming of a mammalian somatic cell, the method comprising:
contacting a population of mammalian somatic cells stem cells with (a) an effective dose of an innate immune response activator; and (b) a cocktail of non-integrating reprogramming or differentiating factors; for a period of time sufficient to reprogram or transdifferentiate said mammalian somatic cells to desired cell type of interest.

2. The method of claim 1, wherein the innate immune response activator and cocktail of non-integrating reprogramming factors are provided simultaneously.

3. The method of claim 1, wherein the innate immune response activator and differentiating factors are provided sequentially.

4. The method of any one of claims 1-4 wherein the innate immune response activator is a Toll-like receptor (TLR) agonist.

5. The method of claim 4, wherein the Toll-like receptor is TLR3.

6. The method of claim 5, wherein the agonist is a double stranded RNA or analog thereof.

7. The method of claim 6, wherein the effective dose of a double stranded RNA or analog thereof is from 10 ng/ml to 3000 ng/ml.

8. The method of claim 4, wherein the Toll-like receptor is TLR4.

9. The method of claim 8, wherein the agonist is a lipopolysaccharide.

10. The method of claim 1, wherein the mammalian somatic cells are human cells.

11. The method of claim 2, wherein the cocktail of reprogramming factors comprises the cell permeant peptides of Oct4, Sox2, Lin28, and Nanog, and the cells are reprogrammed to pluripotency.

12. The method of claim 2, wherein the cocktail of reprogramming factors comprises the cell permeant peptides of Oct4, Sox2, c-Myc, and Klf4, and the cells are reprogrammed to pluripotency.

13. The method of claim 3, wherein the cells are transdifferentiated to a substantially different somatic cell type.

14. The method of claim 8, wherein the substantially different somatic cell type is an endothelial cell.

15. The method of claim 2, wherein the reprogramming factors are provided as cell permeant proteins.

16. A population of induced mammalian pluripotent stem cells produced by the method of claim 2.

17. The population of induced pluripotent stem cells of claim 16, wherein the somatic cells are human cells.

18. A population of transdifferentiated somatic cells produced by the method of claim 3.

19. The population of transdifferentiated somatic cells of claim 18, wherein the cells are human cells.

20. A kit for practicing the method of claim 1.

21. A therapeutic comprising an activator of innate immunity, and one or more cell permeant peptides and/or small molecules, for administration in vivo, for therapeutic modulation of cell and/or tissue phenotype.
